(19) United States
(12) Patent Application Publication
(10) Pub. No.: US 2024/0239750 A1
(43) Pub. Date: Jul. 18, 2024

(54) PHARMACEUTICAL PREPARATION

(71) Applicant: Aerie Pharmaceuticals, Inc., Bedminster, NJ (US)

(72) Inventors: Jonathan Loughrey, East Rutherford, NJ (US); Jaclyn Raeburn, East Rutherford, NJ (US)

(21) Appl. No.: 18/558,974

(22) PCT Filed: May 5, 2022
(86) PCT No.: PCT/US2022/027837

§ 371 Date: Nov. 3, 2023

Related U.S. Application Data
(60) Provisional application No. 63/184,593, filed on May 5, 2021.

(51) Int. Cl. C07D 217/22; A61K 9/00; A61K 31/472; A61K 47/34; C07C 309/30

(52) U.S. Cl. CPC C07D 217/22; A61K 9/0051; A61K 31/472; A61K 47/34; C07C 309/30

(57) ABSTRACT

Provided herein are solid forms of alpha-(aminomethyl)-4-(hydroxymethyl)-N-6-isoquinolinyl-(S)-benzeneacetamide mono-tosylate salt (“Compound 1 mono-tosylate”), pharmaceutical compositions containing the solid forms, methods of producing the solid forms, and methods of treating various ocular diseases or disorders by administering the solid forms.

### RELATED APPLICATIONS

This application claims priority of U.S. Provisional Patent Application No. 63/184,593, filed May 5, 2021, the entire contents of which are hereby incorporated by reference.

### FIELD

The present disclosure relates generally to novel polymorphic forms of alpha-(aminomethyl)-4-(hydroxymethyl)-N-6-isoquinolinyl-(S)-benzeneacetamide mono-tosylate salt (hereinafter referred to as “Compound 1 mono-tosylate”). The disclosure is also directed to pharmaceutical compositions containing at least one polymorphic form of Compound 1 mono-tosylate and to therapeutic or prophylactic use of polymorphic forms and compositions of Compound 1 mono-tosylate.

### BACKGROUND

Compound 1 is a Rho-associated protein kinase inhibitor that is a metabolite of netarsudil. Its chemical structure as a mono-tosylate salt form is set forth below:

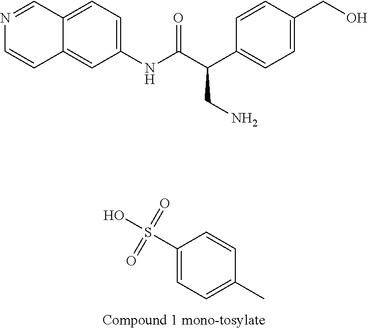 [SMILES: NC[C@@H](C(=O)Nc1ccc2cnccc2c1)c1ccc(CO)cc1 ; Cc1ccc(S(=O)(=O)O)cc1]

Compound 1 is described in U.S. Pat. Nos. 8,394,826, 8,716,310, 951,059, and 10,316,029, and in US Published Patent Applications US20200002324 and US20200131171, all of which are hereby incorporated by reference in their entireties. Compound 1 mono-tosylate is a potent and selective rho-associated kinase inhibitor being developed for treatment of various diseases or disorders, including, but not limited to diabetic eye disease, wet age-related macular degeneration, dry aged-related macular degeneration, inflammation, dry eye, ocular hypertension, an inflammatory disease or disorder, e.g. uveitis, a corneal ulcer, endophthalmitis, an autoimmune disease of the cornea or ocular surface, an ophthalmic manifestation of a viral disease (such as a Coronaviral or Herpes viral disease (i.e. SARS-CoV-2, HIV disease, or ocular herpes)), related diseases of the retina, or any combination of such ocular diseases or disorders.

Rho-kinase is a serine/threonine protein kinase involved in regulation of cell shape and size via action on the cytoskeleton (Hall et al., “RHO GTPases and Actin Cytoskeleton.” Science 279:95350:509-14 (1998)). In particular, these kinases have been identified as having a role to play in determining aqueous humor outflow of the eye via the trabecular pathway, which includes Schlemm's canal, trabecular meshwork and juxtacanalicular tissue ((Alvarado, J., et al., “Age-related Changes in Trabecular Meshwork Cellularity.” Invest. Ophthalmol. Vis. Sci. (21(5):714-27 (1981), (Johnson, M. “What Controls Aqueous Humour Outflow Resistance?”, Exp. Eye Res. 824):545-57 (2006)).

Generally, it is desirable to have crystalline or amorphous forms of a pharmaceutically active compound that possess physical properties amenable to reliable formulation and manufacture. Such properties include filterability, hygroscopicity and flow, as well as stability to heat, moisture and light.

Polymorphs are different crystalline forms of the same compound. The term “polymorph” may include other solid-state molecular forms including hydrates (e.g., bound water present in the crystalline structure) and solvates (e.g., bound solvent other than water) of the same compound. Crystalline polymorphs typically have different crystal structures due to a different packing of the molecules of the crystal lattice. Polymorphs have different crystal symmetries and/or unit cell parameters when compared to each other due to different packing of the molecules in the crystal lattice of each polymorph. As a result, each polymorph has a different crystal symmetry and/or unit cell parameters that directly influence its physical properties such as x-ray diffraction characteristics of crystals or powders.

Polymorphic forms are of interest to the pharmaceutical industry and especially to those involved in the development of suitable dosage forms. If the polymorphic form is not held constant during clinical or stability studies, the exact dosage form used or studied may not be comparable from one lot to another. It is also desirable to have processes for producing a compound with the selected polymorphic form in high purity when the compound is used in clinical studies or commercial products since impurities present may produce undesired toxicological effects. Certain polymorphic forms may also exhibit enhanced thermodynamic stability or may be more readily manufactured in high purity in large quantities. Thus, they are more suitable for inclusion in pharmaceutical formulations. Certain polymorphs may display other advantageous physical properties such as lack of hygroscopic tendencies, improved solubility, improved bioavailability, and enhanced rates of dissolution due to different lattice energies.

Accordingly, what is needed are novel and useful polymorphic crystalline structures of Compound 1 mono-tosylate as well as pharmaceutically acceptable salts thereof.

The discussion of the background disclosure herein is included to explain the context of the present disclosure. It is not to be taken as an admission that any of the material referred therein was published, known or part of the general knowledge in any country as of the priority date of any claims.

The citation of any reference herein should not be deemed as an admission that such reference is available as prior art to the instant disclosure.

### SUMMARY

Provided herein are new and useful heretofore unknown polymorphs of Compound 1 mono-tosylate. Each polymorph of the present disclosure can be uniquely identified by several different analytical parameters, alone or in combination, such as, but not limited to x-ray diffraction pattern (XRPD), differential scanning calorimetry (DSC), Fourier-transform infra-red, or melting point, to name only a few. In particular, the present disclosure is based upon the discovery that, surprisingly and unexpectedly a number of unique and novel crystalline forms of Compound 1 mono-tosylate can be synthesized, isolated and analyzed, and are useful in various processes for the preparation of pharmaceutical compositions comprising Compound 1 mono-tosylate, or one or more of its crystal forms.

Broadly, the present disclosure describes a crystalline form 1 of Compound 1 mono-tosylate. In some embodiments, the crystalline form 1 is characterized by data selected from one or more of the following:
(i) an x-ray powder diffraction (XRPD) pattern having two or more signals selected, independently, from 6.1, 15.8, 18.8, or 23.9 degrees 2θ±0.2 degrees 2θ;
(ii) an XRPD pattern as depicted in FIG. 1;
(iii) an XRPD pattern having two or more signals selected, independently, from 6.1, 15.8, 18.8, or 23.9 degrees 2θ±0.2 degrees 2θ, and having one, two, three, four, five, six, or seven additional signals selected from 5.2, 16.5, 17.8, 19.5, 20.7, 22.8, or 23.1 degrees 2θ±0.2 degrees 2θ; and
(iv) combinations of any of (i)-(iii).

The present disclosure further extends to a crystalline form 2 of Compound 1 mono-tosylate characterized by data selected from one or more of the following:
(i) an x-ray powder diffraction (XRPD) pattern having two or more signals selected, independently, from 5.9, 15.5, or 16.4 degrees 2θ±0.2 degrees 2θ;
(ii) an XRPD pattern as depicted in FIG. 4;
(iii) an XRPD pattern having two or more signals selected, independently, from 5.9, 15.5, or 16.4 degrees 2θ±0.2 degrees 2θ, and having one or two additional signals selected from 5.4 or 21.6 degrees 2θ±0.2 degrees 2θ; and
(iv) combinations of any of (i)-(iii).

The present disclosure further extends to a crystalline form 3 of Compound 1 mono-tosylate characterized by data selected from one or more of the following:
(i) an x-ray powder diffraction (XRPD) pattern having two or more signals selected, independently, from 6.9, 18.1, or 21.8, degrees 2θ±0.2 degrees 2θ;
(ii) an XRPD pattern as depicted in FIG. 18;
(iii) an XRPD pattern having two or more signals selected, independently, from 6.9, 18.1, or 21.8, degrees 2θ±0.2 degrees 2θ, and having one or two additional signals selected from 19.2 or 25.3 degrees 2θ±0.2 degrees 2θ; and
(iv) combinations of any of (i)-(iii).

The present disclosure further extends to a crystalline form 4 of Compound 1 mono-tosylate characterized by data selected from one or more of the following:
(i) an x-ray powder diffraction (XRPD) pattern having two or more signals selected, independently, from 6.0, 17.7, 18.0, or 19.4, degrees 2θ±0.2 degrees 2θ;
(ii) an XRPD pattern as depicted in FIG. 7;
(iii) an XRPD pattern having two or more signals selected, independently, from 6.0, 17.7, 18.0, or 19.4, degrees 2θ±0.2 degrees 2θ, and having one, two, or three additional signals selected from 16.0, 19.7, or 25.2 degrees 2θ±0.2 degrees 2θ; and
(iv) combinations of any of (i)-(iii).

A crystalline form 5 of Compound 1 mono-tosylate is also provided, which is characterized by data selected from one or more of the following:
(i) an x-ray powder diffraction (XRPD) pattern having two or more signals selected, independently, from 6.2, 7.3, 19.8, or 23.4 degrees 2θ±0.2 degrees 2θ;
(ii) an XRPD pattern as depicted in FIG. 10;
(iii) an XRPD pattern having two or more signals selected, independently, from 6.2, 7.3, 19.8, or 23.4 degrees 2θ±0.2 degrees 2θ degrees, and having one, two, three, four, five or six additional signals at 6.6, 10.6, 14.6, 15.9, 21.2, and 27.9 degrees 2θ±0.2 degrees 2θ; and
(iv) combinations of any of (i)-(iii).

In addition, the present disclosure further extends to a crystalline form 6 of Compound 1 mono-tosylate characterized by data selected from one or more of the following:
(i) an x-ray powder diffraction (XRPD) pattern having two or more signals selected, independently, from 9.7, 11.2, 12.8, 14.5, or 23.0 degrees 2θ±0.2 degrees 2θ;
(ii) an XRPD pattern as depicted in FIG. 12;
(iii) an XRPD pattern having two or more signals selected, independently, from 9.7, 11.2, 12.8, 14.5, or 23.0 degrees 2θ±0.2 degrees 2θ, and having one, two, three, or four additional signals selected from 10.9, 20.0, 22.2, or 25.6 degrees 2θ±0.2 degrees 2θ; and
(iv) combinations of any of (i)-(iii).

The present disclosure further extends to a pharmaceutical composition for treating an ocular disease or disorder in a subject. Such a composition comprises a one crystalline form of Compound 1 mono-tosylate and at least one pharmaceutically acceptable excipient. Particular examples of Compound 1 mono-tosylate crystalline forms having applications in a pharmaceutical composition of the disclosure include, but are not limited to crystalline forms 1, 2, 3, 4, 5, or 6 discussed above, as well as any combination thereof.

Such a pharmaceutical composition of the present disclosure can be administered to a subject in need thereof in numerous ways. A particular example having applications in the present disclosure is having a pharmaceutical composition of the present disclosure formulated for intravitreal administration to an eye or both eyes of the subject. A particular formulation for such a pharmaceutical composition of the present disclosure is having the at least one pharmaceutically acceptable excipient in a form of a biodegradable polymer matrix. Optionally, at least one crystalline form of Compound 1 mono-tosylate of the present disclosure can distributed throughout the polymer matrix forming a monolithic biodegradable polymer matrix. Alternatively, a chamber can be formed within a polymer matrix in which at least one crystalline form of Compound 1 mono-tosylate of the present disclosure can be held. In a particular embodiment, a pharmaceutical composition of the present disclosure comprises at least one crystalline form of Compound 1 mono-tosylate and at least one pharmaceutically acceptable excipient in the form of a biodegradable polymer matrix, wherein the at least one crystalline form of Compound 1 mono-tosylate of the present disclosure is distributed homogeneously throughout the biodegradable polymer matrix. In a particular embodiment, a biodegradable polymer matrix of a pharmaceutical composition of the present disclosure is in the form of an ocular implant.

The amount of a biodegradable polymer matrix contained within a pharmaceutical composition of the disclosure generally depends on the biodegradable polymer(s) that form the polymer matrix, the ocular disease or disorder the pharmaceutical composition is intended to treat, the biodegradable polymers that compose the biodegradable polymer matrix, and the desired duration of delivery of a crystalline form of Compound 1 mono-tosylate of the present disclosure. Generally, a pharmaceutical composition of the present disclosure comprises at least about 50 weight % polymer matrix.

In a particular embodiment, a pharmaceutical composition of the present disclosure is formulated to release Compound 1 mono-tosylate in a substantially linear manner for at least about 1 month, 2 months, 3 months, 4 months, 5 months, 6 months, 7 months, 8 months, 9 months, 10 months, 11 months, 12 months, or longer. In a particular embodiment, a pharmaceutical composition of the present disclosure is formulated to release Compound 1 mono-tosylate in a substantially linear manner for about 3 to at least about 6 months.

A biodegradable polymer matrix having applications in a pharmaceutical composition of the present disclosure may optionally comprise a first, a second, a third or even more biodegradable polymers. Optionally, these biodegradable polymers can be mechanically blended with at least one crystalline form of Compound 1 mono-tosylate of the present disclosure to form a pharmaceutical composition of the present disclosure.

In some embodiments, provided herein are implants, comprising at least one solid form of Compound 1 mono-tosylate and a polymer matrix. In some embodiments, an implant may be formed by dry blending any suitable polymer matrix or combination thereof, including, without limitation, those described herein, with at least one solid form of Compound 1 mono-tosylate, and extruding the mixture at a temperature that is above the melting temperature of the polymer and below the melting temperature of the solid form of Compound 1 mono-tosylate to form the ocular implant. In some embodiments, the polymer matrix has a melting temperature that is greater than the temperature of the body in which it is to be implanted, for example, a human or non-human mammal, a livestock, a wild animal, or a pet. In some embodiments, the polymer melting temperature is at least 40° C. In some embodiments, the polymer melting temperature is less than a melting temperature of a Compound 1 mono-tosylate. In some embodiments, the polymer melting temperature is less than a melting temperature range of one or more of a Compound 1 mono-tosylate form as shown in Table 12. In some embodiments, the polymer melting temperature is less than about 114, 123, 182, 183, 184, or 196° C.±5° C. In some embodiments, the polymer melting temperature is less than 114, 123, 182, 183, 184, or 196° C. In some embodiments, the polymer melting temperature is less than about 190° C., less than about 180° C., between about 140-170° C., about 150° C., or between about 110-190° C. In some embodiments, the polymer melting temperature is at least 40° C. and less than about 190° C. In some embodiments, the polymer matrix comprises a polyesteramide (PEA) polymer. In some embodiments, the polymer matrix comprises an amino acid based polyesteramide (i.e. a polyesteramide comprising one or more amino acid side chain moieties).

In some embodiments, the polymer matrix referred to herein may comprise one or more of the polymers described or claimed in U.S. Pat. No. 9,963,549B2, U.S. Pat. No. 10,888,531B2, U.S. Ser. No. 10/624,904B2, WO2017015604A1, US20190083512A1, WO2020181060A1, or US20200306182A1, the entire content of each of which are incorporated herein by reference in their entireties.

In some embodiments, the polymer matrix comprises 25% benzylated polyesteramide or 50% benzylated polyesteramide.

In some embodiments, the polymer matrix comprises a polyphosphazene, a collagen, a polycyanaoacrylate, a poly(lactic acid), a poly(glycolic acid), a gelatin, a poly(hydroxyalkanoate), a cellulose, a polycaprolactone, a polysaccharide (i.e. a chitosan), a polyanhydride, an alginate, a polydioxanone, a starch, an amylose, a polyorthoester, a poly(propylene fumarate), a polyesteramide, a polyamido amine, a polythioester, or a combination thereof. In some embodiments, the polymer matrix comprises a polyphosphazene, a polycyanaoacrylate, a poly(lactic acid), a poly(glycolic acid), a poly(hydroxyalkanoate), a polycaprolactone, a polyanhydride, a polydioxanone, a polyorthoester, a poly(propylene fumarate), a polyesteramide, a polyamido amine, a polythioester, or a combination thereof. In some embodiments, the polymer matrix comprises a collagen, a gelatin, a cellulose, a polysaccharide (i.e. a chitosan), an alginate, a starch, an amylose, a polythioester, or a combination thereof.

In some embodiments, the polymer matrix comprises PEA III X15, PEA III X25, PEA III X50, PEA III Ac Bz, or a combination thereof. In some embodiments, the polymer has an average molecular weight from about 10-80 kDa. In some embodiments, the PEA III Ac Bz comprises 25%, 45%, or 50% mol/mol Bz.

In some embodiments, the polymer matrix comprises one or more of a poly(D,L-lactide) (PLA) having an acid or ester end group, and an inherent viscosity (dL/g) of about 0.15 to 0.75. In some embodiments, the inherent viscosity is about 0.16-0.24, 0.25-0.35, or 0.55-0.75. In some embodiments, the polymer is selected from RESOMER® R 202 H, RESOMER® R 202 S, RESOMER® R 203 H, RESOMER® R 203 S, RESOMER® R 205 S, or a combination thereof. In some embodiments, the polymer can have a molecular weight of about 10,000 to about 28,000.

In some embodiments, the polymer matrix comprises one or more of a poly (D,L-lactide-co-glycolide) (PLGA) having an acid or ester end group, and an inherent viscosity (dL/g) of about 0.09 to 1.7. In some embodiments, the inherent viscosity is about 0.08-0.16, 0.14-0.22, 0.16-0.24, 0.32-0.44, 0.45-0.60, 0.50-0.70, 0.61-0.74, 0.71-1.0, 0.8-1.2, or 0.9-1.3. In some embodiments, the ratio of lactide to glycolide is about 50:50, 65:35, 75:25, or 85:15. In some embodiments, the polymer is selected from RESOMER® RG 501 H, RESOMER® RG 502, RESOMER® RG 502 H, RESOMER® RG 503, RESOMER® RG 503 H, RESOMER® RG 504, RESOMER® RG 504 H, RESOMER® RG 505, RESOMER® RG 653 H, RESOMER® RG 750 S, RESOMER® RG 752 H, RESOMER® RG 752 S, RESOMER® RG 753 H, RESOMER® RG 753 S, RESOMER® RG 755 S, RESOMER® RG 756 S, RESOMER® RG 757 S, RESOMER® RG 858 S, or a combination thereof. In some embodiments, the polymer can have a molecular weight of about 7,000 to about 240,000.

In some embodiments, the polymer matrix comprises polymers of hydroxyaliphatic carboxylic acids, either homopolymers or copolymers, and polysaccharides. In some embodiments, the polymer matrix comprises one or more polyesters. Polyesters may include polymers of D-lactic acid, L-lactic acid, racemic lactic acid, glycolic acid, polycaprolactone, co-polymers thereof, and combinations thereof. Thus, in some embodiments the polymer matrix comprises a synthetic aliphatic polyester, for example, a polymer of lactic acid and/or glycolic acid, and includes poly-(D,L-lactide) (PLA), poly-(D-lactide), poly-(L-lactide), polyglycolic acid (PGA), or the copolymer poly-(D,L-lactide-co-glycolide) (PLGA).

In some embodiments, a biodegradable polymer matrix having applications herein comprises a first biodegradable polymer, a second biodegradable polymer, and optionally even an additional biodegradable polymer or polymers. In some embodiments, the first biodegradable polymer is a polyesteramide polymer, and the second biodegradable polymer is selected from the group consisting of: (i) at least one biodegradable poly(D,L-lactide) polymer, (ii) at least one biodegradable poly (D,L-lactide-co-glycolide) polymer, and (iii) any combination of (i) and (ii). Optionally, such polymers of a pharmaceutical composition of the present disclosure can be ester end-capped or acid-capped.

In a particular embodiment, a biodegradable polymer matrix of a pharmaceutical composition of the present disclosure comprises at least one biodegradable (D,L-lactide) polymer that is an acid end-capped biodegradable poly(D,L-lactide) homopolymer, or an ester end-capped poly(D,L-lactide) homopolymer.

In some embodiments, at least one poly(D,L-lactide-co-glycolide) polymer of a biodegradable polymer matrix of a pharmaceutical composition of the present disclosure can be (i) an ester end-capped biodegradable poly(D,L-lactide-co-glycolide) copolymer, (ii) an acid-capped biodegradable poly(D,L-lactide-co-glycolide) copolymer, or (iii) any combination of (i) and (ii).

A biodegradable polyesteramide homopolymer for use in a biodegradable polymer matrix of a pharmaceutical composition of the disclosure can optionally comprise a polymer structure described in U.S. Ser. No. 10/624,904B2, WO2017015604A1, US20190083512A1, WO2020181060A1, or US20200306182A1.

A particular example of a biodegradable polyesteramide homopolymer having applications in a pharmaceutical composition of the present disclosure comprises the following structure:

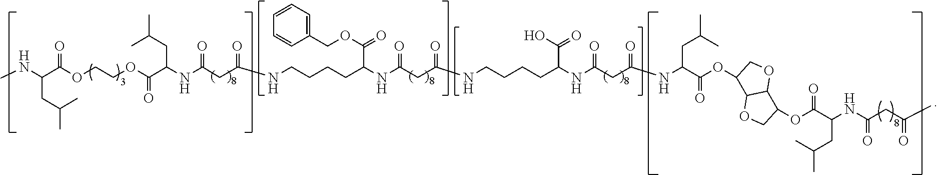

In a particular embodiment, a pharmaceutical composition of the present disclosure is formulated to release a Compound 1 mono-tosylate polymorph disclosed herein in a substantially linear manner for at least about 1 month, at least about 2 months, at least about 3 months, at least about 4 months, at least about 5 months, at least about 6 months, at least 7 months, at least 8 months, at least 9 months, at least 10 months, at least 11 months, at least 12 months, or longer.

The present disclosure further extends to a method of treating an ocular disease or disorder in a subject in need thereof, comprising administering to the subject a therapeutically effective amount of at least one form (e.g., at least one crystalline form) of Compound 1 mono-tosylate to the subject. Particular forms of crystalline of Compound 1 mono-tosylate having applications in such a method of the present disclosure are crystalline forms 1, 2, 4, 5 and 6 as described herein. Such a method of the present disclosure includes administering to the subject a therapeutically effective amount of a pharmaceutical composition of the present disclosure. As explained above, numerous methods of administering a pharmaceutical composition of the present disclosure to the subject are encompassed within the scope of the instant disclosure. In a particular embodiment, such administration is to the vitreous humor of an eye or both eyes of the subject.

Likewise, numerous ocular diseases or disorders can be treated with a method of the disclosure, including those discussed herein, such as glaucoma; a neurodegenerative disease or disorder such as diabetic eye disease, wet age-related macular degeneration, dry aged-related macular degeneration; inflammation, dry eye, ocular hypertension, an inflammatory disease or disorder, e.g. uveitis, a corneal ulcer, endophthalmitis, an autoimmune disease of the cornea or ocular surface, an ophthalmic manifestation of HIV disease, ocular herpes, or any combination of such ocular disease or disorders.

These and other aspects of the present disclosure will be better appreciated by reference to the following drawings and Detailed Description.

### BRIEF DESCRIPTION OF THE DRAWINGS

FIG. 1 shows the x-ray powder diffraction (XRPD) pattern of crystalline form 1 of Compound 1 mono-tosylate.

FIG. 2 shows the Fourier-Transform Infra-Red (FT-IR) spectrum of crystalline form 1 of Compound 1 mono-tosylate obtained from dichloromethane (DCM).

FIG. 3 shows the Thermogravimetric/DSC (TG/DSC) thermogram of crystalline form 1 of Compound 1 mono-tosylate. The thermogram trace is the dashed line, and the DSC trace is the solid line.

FIG. 4 shows the XRPD pattern of crystalline form 2 of Compound 1 mono-tosylate.

FIG. 5 shows the FT-IR spectrum of crystalline form 2 of Compound 1 mono-tosylate obtained from dichloromethane (DCM).

FIG. 6 shows the TG/DSC thermogram of crystalline form 2 of Compound 1 mono-tosylate. The TG trace is the dashed line, and the DSC trace is the solid line.

FIG. 7 shows the XRPD of crystalline form 4 of Compound 1 mono-tosylate.

FIG. 8 shows the FT-IR spectrum of crystalline form 4 of Compound 1 mono-tosylate.

FIG. 9 shows the TG/DSC thermogram of crystalline form 4 of Compound 1 mono-tosylate. The TG trace is the dashed line, and the DSC trace is the solid line.

FIG. 10 shows the XRPD of crystalline form 5 of Compound 1 mono-tosylate.

FIG. 11 shows the TG/DSC thermogram of crystalline form 5 of Compound 1 mono-tosylate. The TG trace is the dashed line, and the DSC trace is the solid line.

FIG. 12 shows the XRPD for crystalline form 6 of Compound 1 mono-tosylate.

FIG. 13 shows the TG/DSC thermogram of crystalline form 6 of Compound 1 mono-tosylate. The TG trace is the dashed line, and the DSC trace is the solid line.

FIG. 14 shows a form diagram for crystalline forms 1-6 of Compound 1 mono-tosylate. i) 2-MeTHF or DCM; ii) DCM post-TC; iii) 1,4-Dioxane; iv) THF; v) 40° C. for 24 h; vi) 40° C./75% RH (open vial) for 7 days; vii) ambient conditions (open vial) for 7 days (partial conversion); viii) saturated ethanol solution with 5% seed added at ambient temperature; ix) slurry in Compound 1 mono-tosylate saturated ethanol containing Patterns 1, 4, and 5 at ambient temperature and 60° C.; x) slurry in Compound 1 mono-tosylate saturated IPA containing Patterns 1, 4, 5, and 6 at ambient temperature; TC: thermal cycling.

FIG. 15 shows the XRPD of amorphous Compound 1 mono-tosylate.

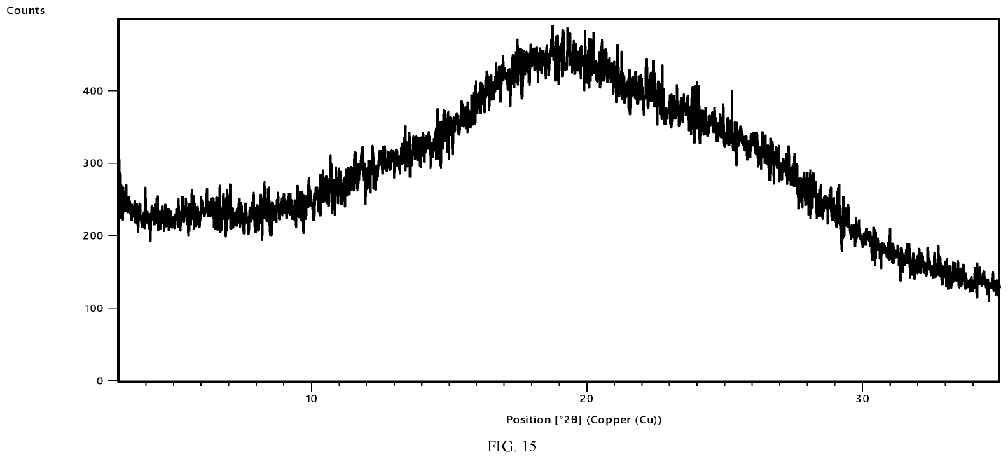

FIG. 16 shows the FT-IR spectrum of amorphous Compound 1 mono-tosylate.

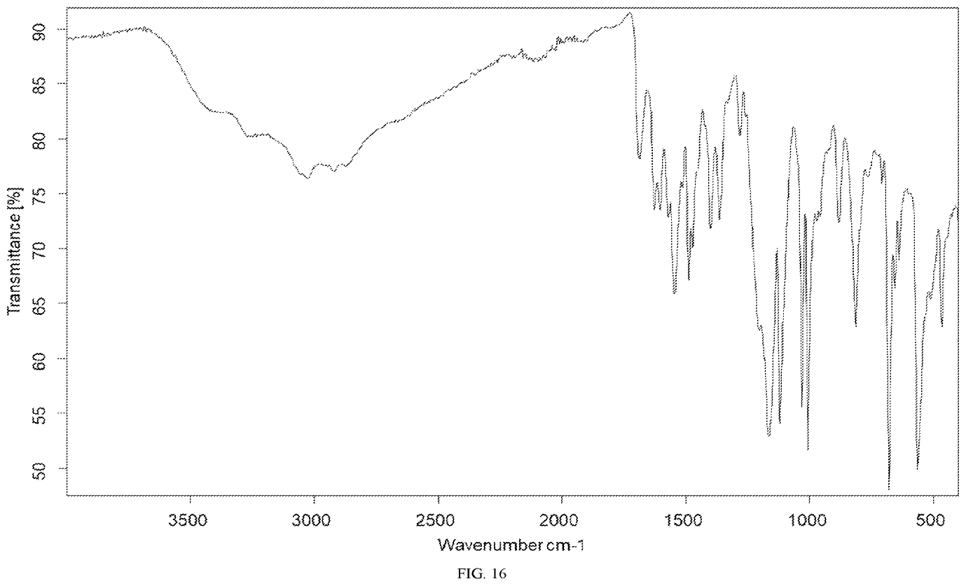

FIG. 17 shows the TG/DSC thermogram of amorphous Compound 1 mono-tosylate. The TG trace is the dashed line, and the DSC trace is the solid line.

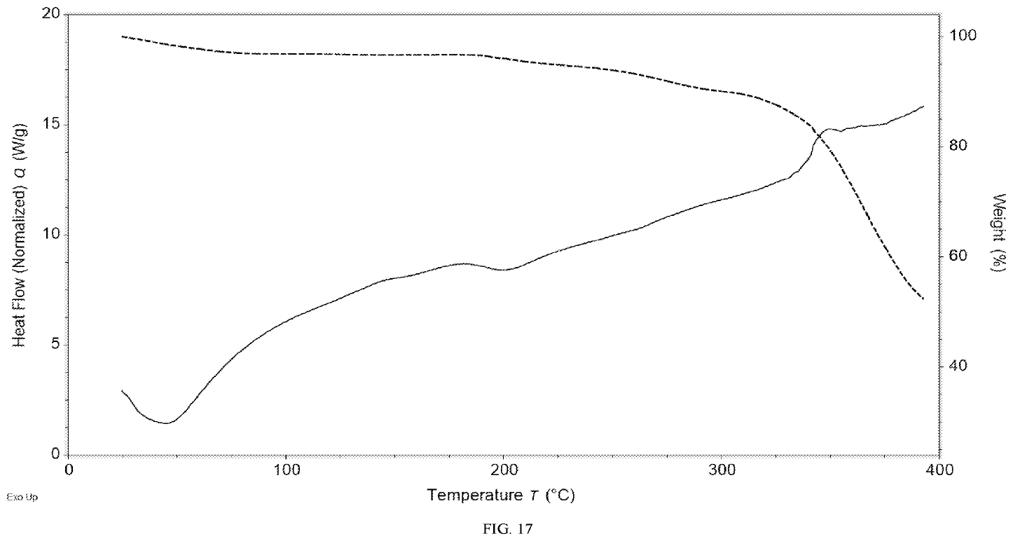

FIG. 18 shows the XRPD pattern of crystalline form 3 of Compound 1 mono-tosylate.

### DETAILED DESCRIPTION

The present disclosure is based upon the discovery that surprisingly and unexpectedly, solid forms (e.g., crystalline forms) of Compound 1 mono-tosylate can be successfully prepared and isolated. The crystalline forms of Compound 1 mono-tosylate of the present disclosure may have advantageous properties relative to those of amorphous Compound 1 mono-tosylate. Examples of such advantageous properties include, but are certainly not limited to, chemical or polymorphic purity; flowability; solubility; dissolution rate; bioavailability; morphology or crystal habit; a lower degree of hygroscopicity, a low content of residual solvents, advantageous processing and handling characteristics such as compressibility or bulk density; and stability, e.g., chemical stability, thermal and mechanical stability with respect to polymorphic conversion, and stability towards dehydration and/or storage stability, to name only a few such properties.

Thus, in some embodiments, provided herein are solid forms of Compound 1 mono-tosylate.

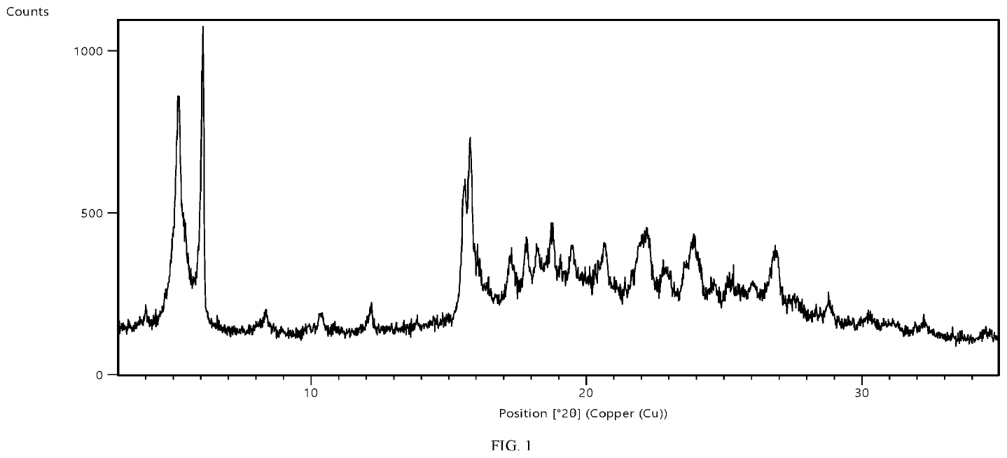

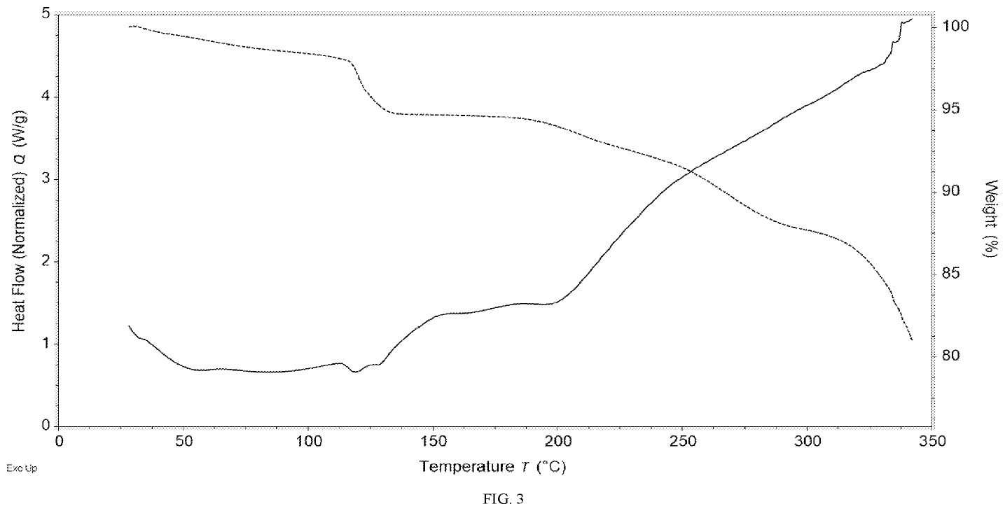

In some embodiments, the solid form is form 1 of Compound 1 mono-tosylate. In some embodiments, form 1 has an XRPD pattern comprising one or more signals, in terms of 20, selected from signals at about 6.1, 15.8, 18.8, or 23.9±0.2 degrees 2θ. In some embodiments, form 1 has an XRPD pattern further comprising one or more signals, in terms of 20, selected from signals at about 5.2, 16.5, 17.8, 19.5, 20.7, 22.8, or 23.1±0.2 degrees 2θ. In some embodiments, form 1 has an XRPD pattern comprising three or more signals, in terms of 20, selected from signals at about 5.2, 6.1, 15.8, 16.5, 17.8, 18.8, 19.5, 20.7, 22.8, 23.1, or 23.9±0.2 degrees 2θ. In some embodiments, form 1 has an XRPD pattern substantially as shown in FIG. 1. In some embodiments, form 1 has a differential scanning calorimetry (DSC) thermogram comprising one or more endothermic signals selected from about 52.9, 128.9, and 201.3° C. In some embodiments, form 1 has a DSC thermogram substantially as shown in FIG. 3. In some embodiments, form 1 has a thermogravimetric analysis (TGA) substantially as shown in FIG. 3. In some embodiments, form 1 has a melting point range of about 114-131° C. In some embodiments, provided herein are compositions comprising form 1. In some embodiments, provided herein are pharmaceutical compositions comprising form 1, and a pharmaceutically acceptable carrier. In some embodiments, form 1 is present in the composition or pharmaceutical composition in an amount of at least about 50%, 60%, 70%, 80%, or 90% by weight. In some embodiments, form 1 is present in the composition or pharmaceutical composition in an amount of about 51% by weight. In some embodiments, the composition consists essentially of form 1. In some embodiments, form 1 is substantially purified. In some embodiments, form 1 is crystalline.

Also provided herein are processes for preparing form 1, comprising precipitating form 1 from a solution comprising Compound 1 mono-tosylate and 2-MeTHF or DCM, optionally including thermal cycling to facilitate the precipitation. In some embodiments, provided herein is a solid form of Compound 1 mono-tosylate prepared by these processes.

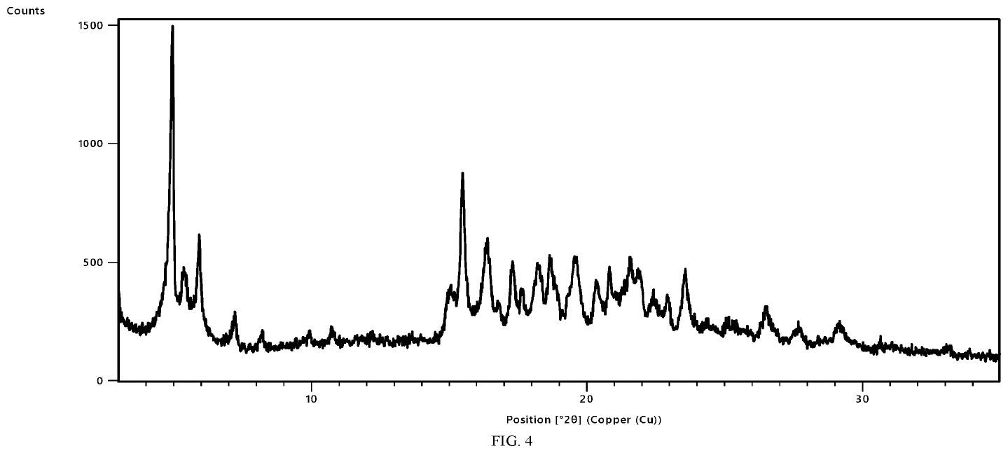

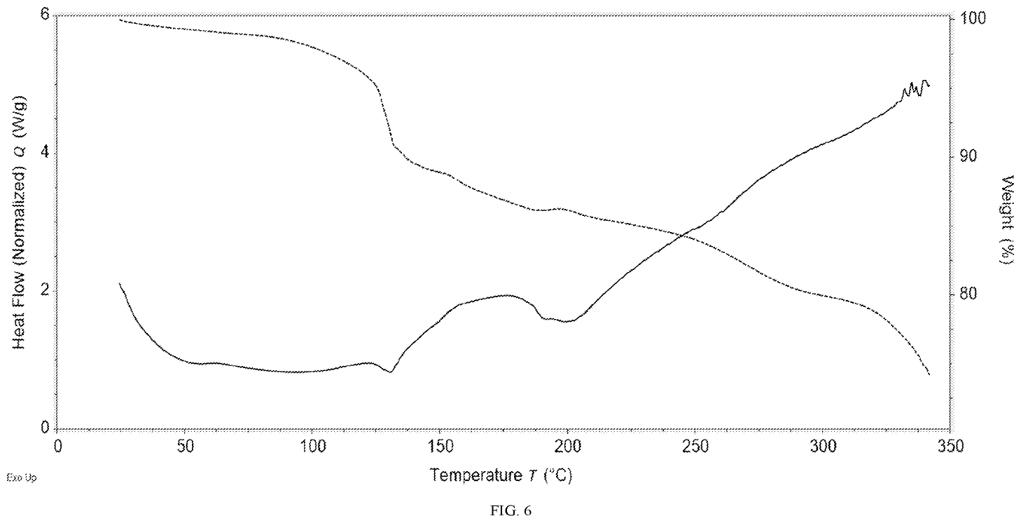

In some embodiments, the solid form is form 2 of Compound 1 mono-tosylate. In some embodiments, form 2 has an XRPD pattern comprising one or more signals, in terms of 20, selected from signals at about 5.9, 15.5, or 16.4±0.2 degrees 2θ. In some embodiments, form 2 has an XRPD pattern further comprising one or more signals, in terms of 20, selected from signals at about 5.4 or 21.6±0.2 degrees 2θ. In some embodiments, form 2 has an XRPD pattern comprising three or more signals, in terms of 20, selected from signals at about 5.4, 5.9, 15.5, 16.4, or 21.6±0.2 degrees 2θ. In some embodiments, form 2 has an XRPD pattern substantially as shown in FIG. 4. In some embodiments, form 2 has a differential scanning calorimetry (DSC) thermogram comprising one or more endothermic signals selected from about 131.0 and 199.7° C. In some embodiments, form 2 has a DSC thermogram substantially as shown in FIG. 6. In some embodiments, form 2 has a thermogravimetric analysis (TGA) substantially as shown in FIG. 6. In some embodiments, form 2 has a melting point range of about 123-131° C. In some embodiments, provided herein are compositions comprising form 2. In some embodiments, provided herein are pharmaceutical compositions comprising form 2, and a pharmaceutically acceptable carrier. In some embodiments, form 2 is present in the composition or pharmaceutical composition in an amount of at least about 50%, 60%, 70%, 80%, or 90% by weight. In some embodiments, form 2 is present in the composition or pharmaceutical composition in an amount of about 51% by weight. In some embodiments, the composition consists essentially of form 2. In some embodiments, form 2 is substantially purified. In some embodiments, form 2 is crystalline.

In some embodiments of the solid forms provided herein, the solid form is a hemi-hydrate or mono-dioxane solvate of Compound 1 mono-tosylate. In some embodiments of the solid forms provided herein, the solid form is a crystalline hemi-hydrate or mono-dioxane solvate of Compound 1 mono-tosylate.

Also provided herein are processes for preparing form 2, comprising precipitating form 2 from a solution comprising Compound 1 mono-tosylate and 1,4-Dioxane, optionally including thermal cycling to facilitate the precipitation. In some embodiments, provided herein is a solid form of Compound 1 mono-tosylate prepared by these processes.

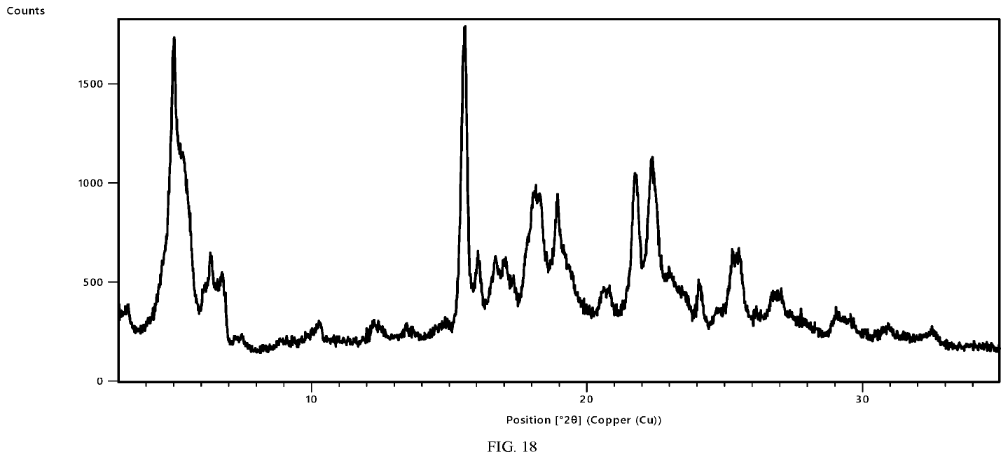

In some embodiments, the solid form is form 3 of Compound 1 mono-tosylate. In some embodiments, form 3 has an XRPD pattern comprising one or more signals, in terms of 20, selected from signals at about 6.9, 18.1, or 21.8±0.2 degrees 2θ. In some embodiments, form 3 has an XRPD pattern further comprising one or more signals, in terms of 20, selected from signals at about 19.2 or 25.3±0.2 degrees 2θ. In some embodiments, form 3 has an XRPD pattern comprising three or more signals, in terms of 20, selected from signals at about 6.9, 18.1, 19.2, 21.8, or 25.3±0.2 degrees 2θ. In some embodiments, form 3 has an XRPD pattern substantially as shown in FIG. 18. In some embodiments, provided herein are compositions comprising form 3. In some embodiments, provided herein are pharmaceutical compositions comprising form 3, and a pharmaceutically acceptable carrier. In some embodiments, form 3 is present in the composition or pharmaceutical composition in an amount of at least about 50%, 60%, 70%, 80%, or 90% by weight. In some embodiments, form 3 is present in the composition or pharmaceutical composition in an amount of about 51% by weight. In some embodiments, the composition consists essentially of form 3. In some embodiments, form 3 is substantially purified. In some embodiments, form 3 is crystalline.

Also provided herein are processes for preparing form 3, comprising precipitating form 3 from a solution comprising Compound 1 mono-tosylate and THF, optionally including thermal cycling to facilitate the precipitation. In some embodiments, provided herein is a solid form of Compound 1 mono-tosylate prepared by these processes.

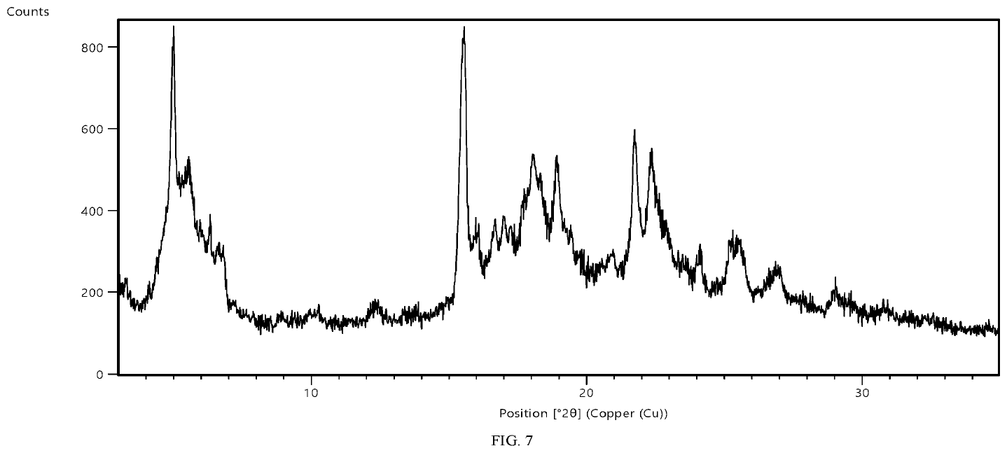

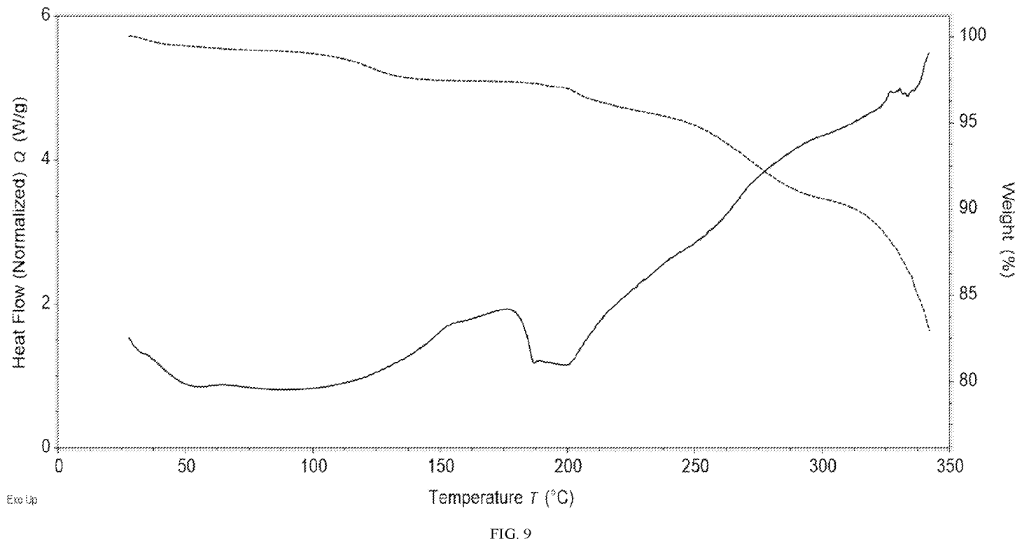

In some embodiments, the solid form is form 4 of Compound 1 mono-tosylate. In some embodiments, form 4 has an XRPD pattern comprising one or more signals, in terms of 20, selected from signals at about 6.0, 17.7, 18.0, or 19.4±0.2 degrees 2θ. In some embodiments, form 4 has an XRPD pattern further comprising one or more signals, in terms of 20, selected from signals at about 16.0, 19.7, or 25.2±0.2 degrees 2θ. In some embodiments, form 4 has an XRPD pattern comprising three or more signals, in terms of 20, selected from signals at about 6.0, 16.0, 17.7, 18.0, 19.4, 19.7, or 25.2±0.2 degrees 2θ. In some embodiments, form 4 has an XRPD pattern substantially as shown in FIG. 7. In some embodiments, form 4 has a differential scanning calorimetry (DSC) thermogram comprising one or more endothermic signals at about 115.3 and 199.9° C. In some embodiments, form 4 has a DSC thermogram substantially as shown in FIG. 9. In some embodiments, form 4 has a thermogravimetric analysis (TGA) substantially as shown in FIG. 9. In some embodiments, form 4 has a melting point range of about 182-200° C. In some embodiments, provided herein are compositions comprising form 4. In some embodiments, provided herein are pharmaceutical compositions comprising form 4, and a pharmaceutically acceptable carrier. In some embodiments, form 4 is present in the composition or pharmaceutical composition in an amount of at least about 50%, 60%, 70%, 80%, or 90% by weight. In some embodiments, form 4 is present in the composition or pharmaceutical composition in an amount of about 51% by weight. In some embodiments, the composition consists essentially of form 4. In some embodiments, form 4 is substantially purified. In some embodiments, form 4 is crystalline.

Also provided herein are processes for preparing form 4, comprising precipitating form 4 from a solution comprising Compound 1 mono-tosylate and THF, and drying the precipitate for more than two hours (i.e. for 16 hours or 24 hours) at about 40° C., optionally including thermal cycling to facilitate the precipitation. In some embodiments, provided herein is a solid form of Compound 1 mono-tosylate prepared by these processes.

In some embodiments, the solid form is form 5 of Compound 1 mono-tosylate. In some embodiments, form 5 has an XRPD pattern comprising one or more signals, in terms of 20, selected from signals at about 6.2, 7.3, 19.8, or 23.4±0.2 degrees 2θ. In some embodiments, form 5 has an XRPD pattern further comprising one or more signals, in terms of 20, selected from signals at about 6.6, 10.6, 14.6, 15.9, 21.2, and 27.9±0.2 degrees 2θ. In some embodiments, form 5 has an XRPD pattern comprising three or more signals, in terms of 20, selected from signals at about 6.2, 6.6, 7.3, 10.6, 14.6, 15.9, 16.2, 17.9, 18.6, 19.8, 20.2, 21.2, 22.6, 23.4, 25.4, 25.7, 27.9, or 28.4±0.2 degrees 2θ.

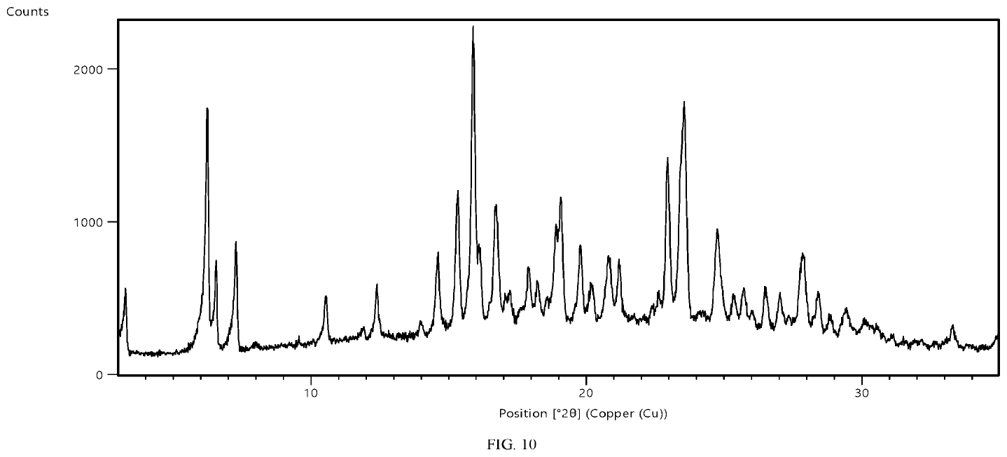

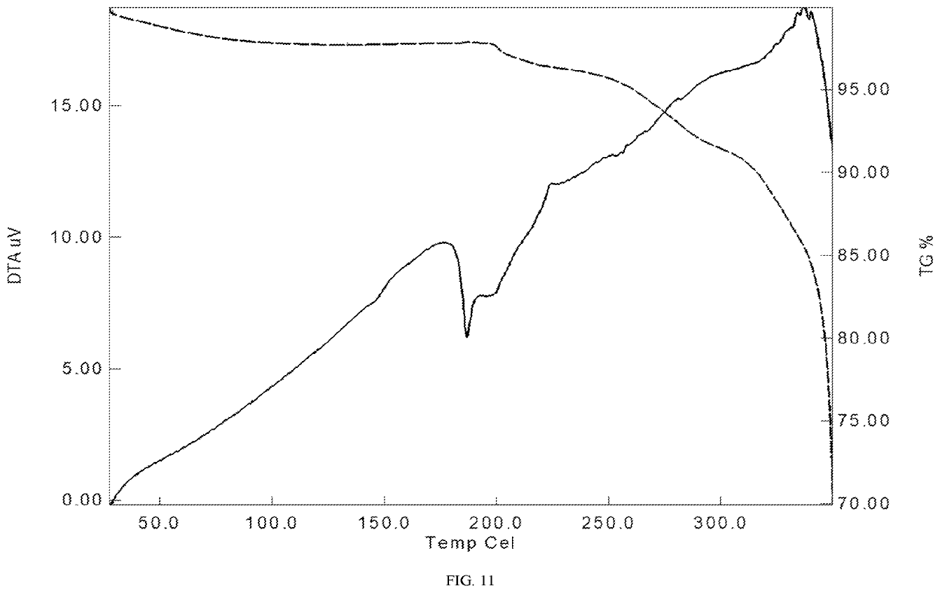

In some embodiments, form 5 has an XRPD pattern substantially as shown in FIG. 10. In some embodiments, form 5 has a differential scanning calorimetry (DSC) thermogram comprising an endothermic signal at about 186.8, 198.7, and 223.8° C. In some embodiments, form 5 has a DSC thermogram substantially as shown in FIG. 11. In some embodiments, form 5 has a thermogravimetric analysis (TGA) substantially as shown in FIG. 11. In some embodiments, form 5 has a melting point range of about 183-199° C. In some embodiments, provided herein are compositions comprising form 5. In some embodiments, provided herein are pharmaceutical compositions comprising form 5, and a pharmaceutically acceptable carrier. In some embodiments, form 5 is present in the composition or pharmaceutical composition in an amount of at least about 50%, 60%, 70%, 80%, or 90% by weight. In some embodiments, form 5 is present in the composition or pharmaceutical composition in an amount of about 51% by weight. In some embodiments, the composition consists essentially of form 5. In some embodiments, form 5 is substantially purified. In some embodiments, form 5 is crystalline.

Also provided herein are processes for preparing form 5, comprising storing form 4 of Compound 1 mono-tosylate at 40° C. and 75% relative humidity for 7 days. In some embodiments, form 5 is prepared by hydration (in some embodiments, hemi-hydration) of amorphous Compound 1 mono-tosylate). In some embodiments, provided herein is a solid form of Compound 1 mono-tosylate prepared by these processes.

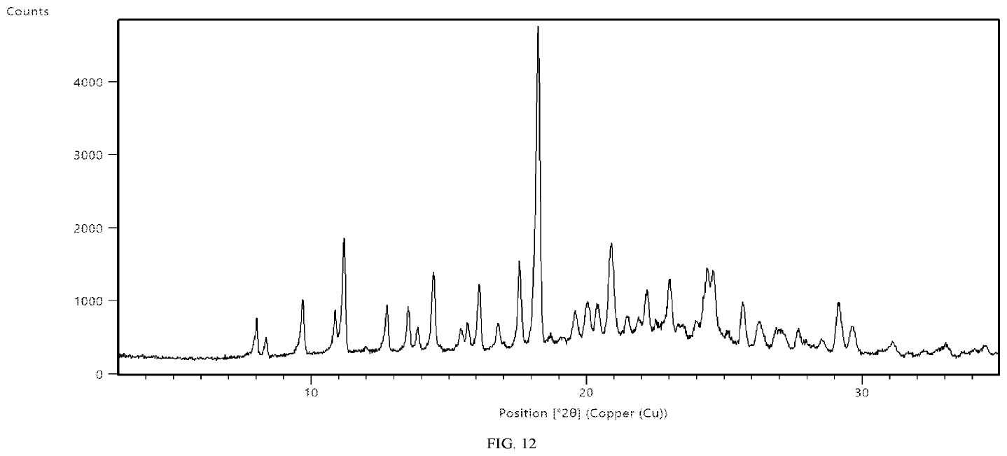

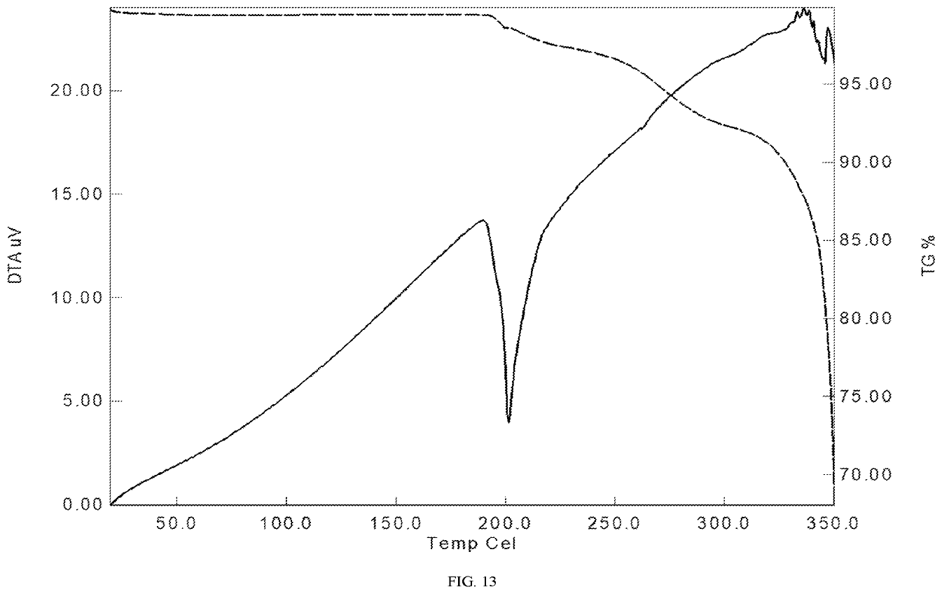

In some embodiments, the solid form is form 6 of Compound 1 mono-tosylate. In some embodiments, form 6 has an XRPD pattern comprising one or more signals, in terms of 20, selected from signals at about 9.7, 11.2, 12.8, 14.5, or 23.0±0.2 degrees 2θ. In some embodiments, form 6 has an XRPD pattern further comprising one or more signals, in terms of 20, selected from signals at about 10.9, 20.0, 22.2, or 25.6±0.2 degrees 2θ. In some embodiments, form 6 has an XRPD pattern comprising three or more signals, in terms of 20, selected from signals at about 9.7, 10.9, 11.2, 12.8, 14.5, 20.0, 22.2, 23.0, or 25.6±0.2 degrees 2θ. In some embodiments, form 6 has an XRPD pattern substantially as shown in FIG. 12. In some embodiments, form 6 has a differential scanning calorimetry (DSC) thermogram comprising an endothermic signal at about 201.5° C. In some embodiments, form 6 has a DSC thermogram substantially as shown in FIG. 13. In some embodiments, form 6 has a thermogravimetric analysis (TGA) substantially as shown in FIG. 13. In some embodiments, form 6 has a melting point range of about 196-202° C. In some embodiments, provided herein are compositions comprising form 6. In some embodiments, provided herein are pharmaceutical compositions comprising form 6, and a pharmaceutically acceptable carrier. In some embodiments, form 6 is present in the composition or pharmaceutical composition in an amount of at least about 50%, 60%, 70%, 80%, or 90% by weight. In some embodiments, form 6 is present in the composition or pharmaceutical composition in an amount of about 51% by weight. In some embodiments, the composition consists essentially of form 6. In some embodiments, form 6 is substantially purified. In some embodiments, form 6 is crystalline.

Also provided herein are processes for preparing form 6, comprising precipitating form 6 from a solution comprising Compound 1 mono-tosylate and saturated ethanol. In some embodiments, form 6 is prepared from one or more of forms 1, 4, or 5 at 25° C. in saturated ethanol. In some embodiments, form 6 is prepared from one or more of forms 1, 4, or 5 at 60° C. in saturated ethanol. In some embodiments, form 6 is prepared from a slurry of forms 1, 4, and 5 at 25° C. in saturated ethanol. In some embodiments, form 6 is prepared from a slurry of forms 1, 4, and 5 at 60° C. in saturated ethanol. In some embodiments, form 6 is prepared from one or more of forms 1, 4, or 5 at 25° C. in saturated isopropyl alcohol. In some embodiments, form 6 is prepared from a slurry of forms 1, 4, 5, 6 at 25° C. in saturated isopropyl alcohol. In some embodiments, provided herein is a solid form of Compound 1 mono-tosylate prepared by these processes.

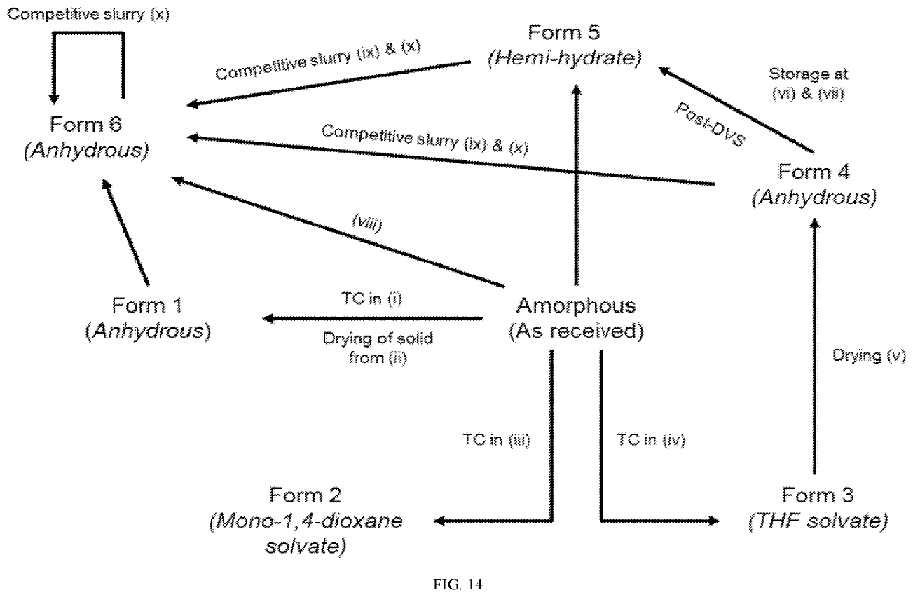

In some embodiments, provided herein is a solid form of Compound 1 mono-tosylate prepared as described by one or more of the processes described in FIG. 14.

In some embodiments, the solid form has an XRPD pattern substantially as shown in FIG. 1, FIG. 4, FIG. 18, FIG. 7, FIG. 10, or FIG. 12.

In some embodiments, the solid form has a differential scanning calorimetry (DSC) thermogram substantially as shown in FIG. 3, FIG. 6, FIG. 9, FIG. 11, or FIG. 13.

In some embodiments, the solid form has a thermogravimetric analysis (TGA) substantially as shown in FIG. 3, FIG. 6, FIG. 9, FIG. 11, or FIG. 13.

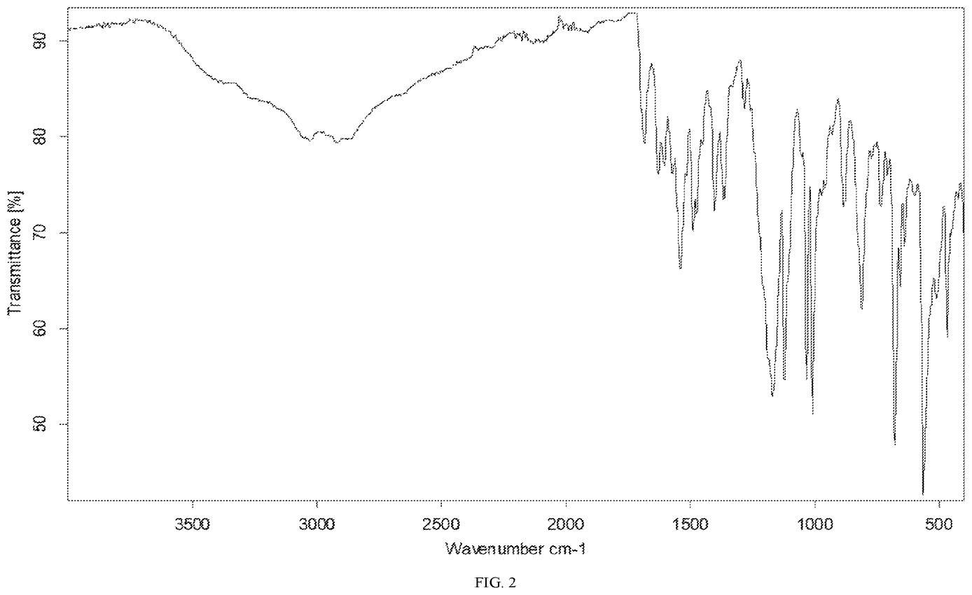

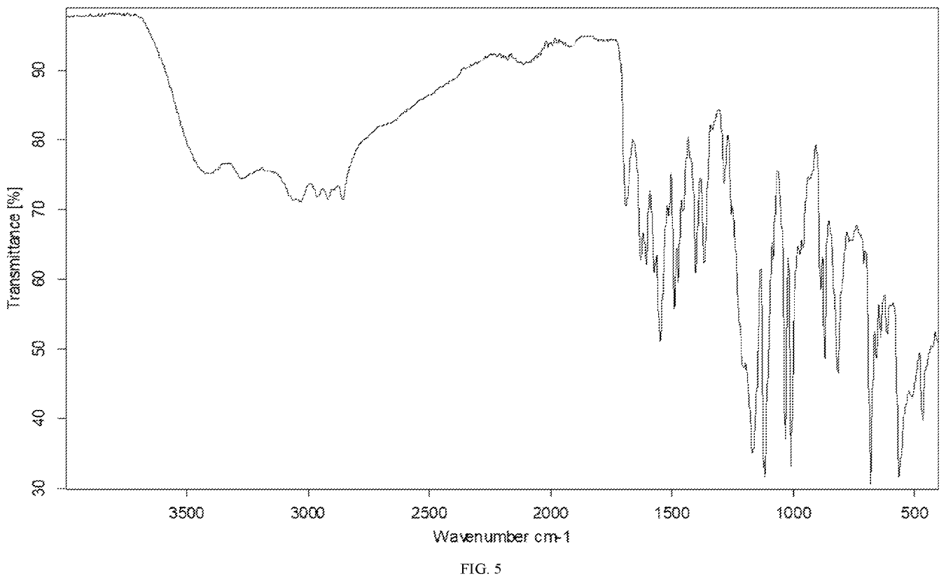

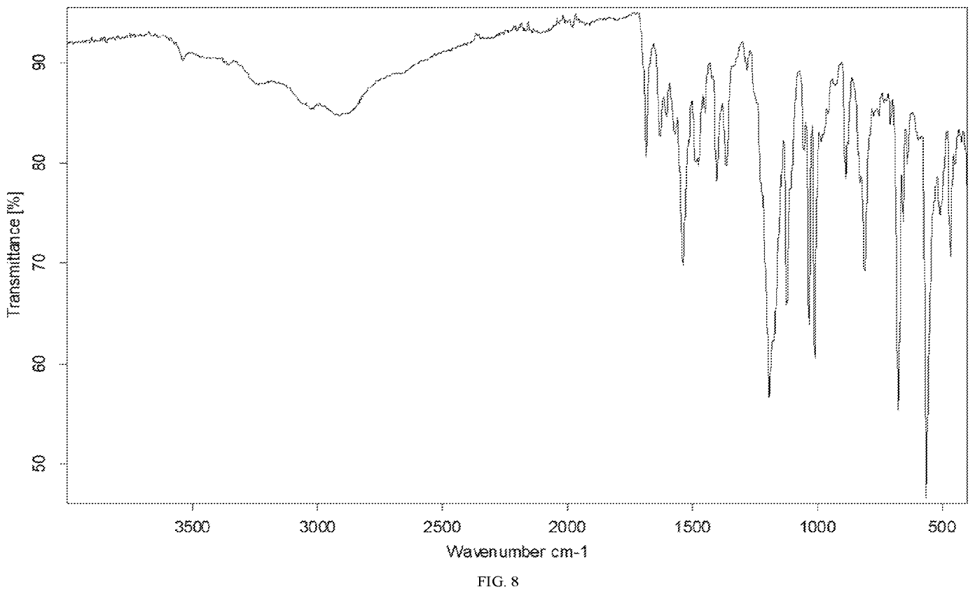

In some embodiments, the solid form has a Fourier-transform infra-red (FT-IR) spectrum substantially as shown in FIG. 2, FIG. 5, or FIG. 8.

Also provided herein are methods of treating an ocular disease or disorder in a subject in need thereof, comprising administering to the subject a therapeutically effective amount of one or more of the solid forms of Compound 1 mono-tosylate provided herein, a composition thereof, or a pharmaceutical composition thereof. In some embodiments, the solid form is form 1, form 2, form 3, form 4, form 5, or form 6, or a combination thereof. In some embodiments, administering to the subject comprises administering to the vitreous humor of an eye of the subject. In some embodiments, the ocular disease or disorder comprises glaucoma, a neurodegenerative disease or disorder, ocular hypertension, an inflammatory disease or disorder, or any combination thereof. In some embodiments, the neurodegenerative disease or disorder comprises diabetic eye disease, wet age-related macular degeneration, dry aged-related macular degeneration, inflammation, dry eye, or any combination thereof. In some embodiments, the inflammatory disease or disorder comprises uveitis, a corneal ulcer, endophthalmitis, an autoimmune disease of the cornea or ocular surface, an ophthalmic manifestation of HIV disease, ocular herpes. In some embodiments, the ocular disease or disorder comprises diabetic eye disease, wet age-related macular degeneration, dry aged-related macular degeneration, inflammation, dry eye, ocular hypertension, an inflammatory disease or disorder, e.g. uveitis, a corneal ulcer, endophthalmitis, an autoimmune disease of the cornea or ocular surface, an ophthalmic manifestation of a viral disease (such as a Coronaviral or Herpes viral disease (i.e. SARS-CoV-2, HIV disease, or ocular herpes)), related diseases of the retina, or any combination of such ocular diseases or disorders.

A crystalline form of Compound 1 mono-tosylate of the present disclosure may be referred to herein as being characterized by graphical data, such as powder X-ray diffractograms (XRPD), solid state ¹H NMR spectra, Fourier-transform infra-red (FT-IR) spectra, thermogravimetric/differential scanning calorimetry (TG/DSC) spectra and PLM light micrographs. As is well known in the art, the graphical data potentially provides additional technical information to further define the respective crystalline form (a so-called “fingerprint”) that cannot necessary be described by reference to numerical values or signal positions alone. Regardless, a person of ordinary skill sill readily understands that such graphical representations of data may be subject to small variations, e.g., in signal relative intensities and signal positions due to factors such as variations in instrument response and variations in sample concentration and purity, which are well known to the skilled person. Nonetheless, one or ordinary skill in the art is readily capable of comparing the graphical data in the figures herein with graphical data generated for an unknown crystal form and confirming whether the two sets of graphical data are characterizing the same crystalline form or two different crystalline forms. Thus, a crystalline form of Compound 1 mono-tosylate disclosed herein and characterized by graphical data “as depicted in” a figure will thus be understood to include any crystalline forms of Compound 1 mono-tosylate characterized with graphical data having such small variations, as are well known to the skilled person, in comparison with the figure.

A crystalline form (aka polymorph) of Compound 1 mono-tosylate may be referred to herein as polymorphically pure or substantially free of any other solid state (or polymorphic) forms. As used herein, the phrase “substantially free of any other forms” will be understood to mean that the solid state form contains about 20% or less, about 10% or less, about 5% or less, about 2% or less, about 1% or less, or about 0% of any other forms of the subject compound as measured, for example, by XRPD. Thus, a crystalline form of Compound 1 mono-tosylate described herein substantially free of any other crystalline forms would be understood to contain greater than about 80% (w/w), greater than about 90% (w/w), greater than about 95% (w/w), greater than about 98% (w/w), greater than about 99% (w/w), or about 100% (w/w) of the subject solid form (e.g., crystalline form) of Compound 1 mono-tosylate. Accordingly, in some embodiments of the present disclosure, a crystalline form of Compound 1 mono-tosylate may contain from about 1% to about 20% (w/w), from about 5% to about 20% (w/w), or from about 5% to about 10% (w/w) of one or more other crystalline forms of Compound 1 mono-tosylate.

Powders analyzed by XRPD spectroscopy may include components other than the crystalline compound meant to be identified, which may result in signals present in an XRPD diffractogram in addition to those attributed to the crystalline compound to be identified. Two or more adjacent or overlapping signals may also result from a particular compound. Thus, in some embodiments, an XRPD signal may present as a component of a broadened peak, a shouldered peak, or a split peak, which result from two or more overlapping or adjacent signals. In some embodiments, an XRPD signal may be synonymous with an XRPD peak.

As used herein, unless stated otherwise, XRPD signals reported herein are preferably measured using CuK α radiation, λ=1.5418 Å.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure.

It is also stated that the reagents described herein are merely exemplary and that equivalents of such are known in the art.

Numerous terms and phrases are used throughout the instant specification and claims and are defined below.

As used herein, the singular form of “a”, “an” and “the” include plural references unless the context clearly dictates otherwise.

As used herein, the term “comprising” is intended to mean that the compositions and methods include the recited elements, but do not exclude others.

“About” and “approximately” are interchangeable, and mean plus or minus a percent (e.g., +5%) of the number, parameter, or characteristic so qualified, which would be understood as appropriate by a skilled artisan to the scientific context in which the term is utilized. Furthermore, since all numbers, values, and expressions referring to quantities used herein, are subject to the various uncertainties of measurement encountered in the art, and then unless otherwise indicated, all presented values may be understood as modified by the term “about.”

Where a numerical range is disclosed herein, then such a range is continuous, inclusive of both the minimum and maximum values of the range, as well as every value between such minimum and maximum values. Still further, where a range refers to integers, every integer between the minimum and maximum values of such range is included. In addition, where multiple ranges are provided to describe a feature or characteristic, such ranges can be combined. That is to say that, unless otherwise indicated, all ranges disclosed herein are to be understood to encompass any and all subranges subsumed therein. For example, a stated range of from “1 to 10” should be considered to include any and all subranges between the minimum value of 1 and the maximum value of 10.

As used herein, “active pharmaceutical ingredient” (API), and “therapeutic agent” can be used interchangeably and refer to a compound or substance in a pharmaceutical composition that is biologically active and produces the effects of the pharmaceutical composition. Examples of APIs described herein are the polymorphic forms of Compound 1 mono-tosylate of the present disclosure.

As used herein, the term “anti-solvent” refers to a solvent in which a compound that is desired to be isolated is less solvent in than the solvent in which the compound is presently dissolved. An anti-solvent is used in crystalline purification in order to induce precipitation of the desired compound.

As used herein, the term “pharmaceutical composition” refers to a composition that comprises an API, excipient, a carrier, etc. Generally, pharmaceutical compositions are administered to a patient rather than the API alone.

As used herein, the phrase “ocular disease or disorder” includes, but is not limited to glaucoma, allergy, inflammatory eye diseases or disorders, ocular hypertension, cancers of the eye, neurodegenerative diseases or disorders of the eye such as diabetic macular edema (DME) and wet or dry age-related macular degeneration (AMD), uveitis, diabetic retinopathy, and dry eye, to name only a few.

As used herein, “kinase” is a type of enzyme that transfers a phosphate group from a high-energy donor, such as ATP, to a specific target molecule (substrate). The process is called phosphorylation.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located.

Typically, room temperature is from about 20° C. to about 30° C., or about 22° C. to about 27° C., or about 25° C. A process or step may be referred to herein as being carried out “overnight.” This refers to a time interval, e.g., for the process or step, that spans the time during the night, when that process or step may not be actively observed. This time interval is from about 8 to about 20 hours, or about 10 to about 18 hours, typically about 16 hours.

Unless otherwise indicated, as used herein, the term “solvate” refers to a crystal form that incorporates a solvent in the crystal structure. When the solvent is water, the solvate is often referred to as a “hydrate.” The solvent in a solvate may be present in either a stoichiometric or in a non-stoichiometric amount.

The term “treatment” refers to the application of one or more specific procedures used for the amelioration of a disease or a disorder. In certain embodiments, the specific procedure is the administration of one or more pharmaceutical agents. “Treatment” of an individual (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Treatment includes any desirable effect on the symptoms or pathology of a disease or disorder, and may include, for example, minimal changes or improvements in one or more measurable markers of the disease or disorder being treated. Also included are “prophylactic” treatments, which can be directed to reducing the rate of progression of the disease or condition being treated, delaying the onset of that disease or condition, or reducing the severity of its onset.

An “effective amount” or “therapeutically effective amount” refers to an amount of therapeutic compound, such as any of the solid forms of Compound 1 mono-tosylate provided herein, administered to a subject (i.e. a mammalian subject), either as a single dose or as part of a series of doses, which is effective to produce a desired therapeutic effect.

The term “amelioration” means a lessening of severity of at least one indicator of a disease or disorder. In certain embodiments, amelioration includes a delay or slowing in the progression of one or more indicators of a disease or disorder. The severity of indicators may be determined by subjective or objective measures known to those skilled in the art.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding methyl tert-butyl ether (MTBE) (1.5 v/v) to a 100 ml reaction mixture would indicate that 150 mL of MTBE was added.

As used herein, “birefringence” refers to an optical property of a material having a refractive index that depends on the polarization and propagation direction of light. Birefringence is responsible for the phenomenon of double refraction whereby a ray of light, when incident upon a birefringent material, is split by polarization into two rays taking slightly different paths. Crystals generally display birefringence.

The term “fingerprint region” in reference to an IR spectrum generally refers to the region of the spectrum between 400 cm⁻¹ and 1500 cm⁻¹. This region usually contains a large number of signals, making it difficult to identify individual signals. However, the fingerprint region of a given compound is unique and, therefore, can be used to distinguish between compounds. For Compound 1 mono-tosylate, the fingerprint range of its IR spectrum ranges from about 450 cm⁻¹ to about 1700 cm⁻¹.

The present disclosure extends to processes for preparing crystalline forms of Compound 1 mono-tosylate. The process comprises preparing amorphous Compound 1 mono-tosylate, and then converting it to various crystalline forms. Discussions regarding the processes for preparing crystalline forms of Compound 1 mono-tosylate of the present disclosure are discussed in the Examples set forth infra.

Having described the present disclosure with reference to certain particular embodiments, other embodiments will become apparent to one skilled in the art from consideration of the instant specification. The present disclosure is further illustrated by reference to the following non-limiting examples describing in detail the preparation of the composition and methods of use of the present disclosure. It will be apparent to those of ordinary skill in the art that many modifications, both to materials and methods, may be practiced without departing from the scope and spirit of the present disclosure.

The present disclosure may be better understood by reference to the following non-limiting examples, which are provided as exemplary of the disclosure. The following examples are presented in order to more fully illustrate the particular embodiments of the disclosure. They should in no way be construed, however, as limiting the broad scope of the disclosure.

### EXAMPLES

### Description of Analyses Performed

X-Ray Powder Diffraction Method: All XRPD analyses described herein were carried on a PANalytical X'pert pro with PIXcel detector (128 channels), scanning the samples between 3 and 35° 20. The material to be analyzed was gently ground to release any agglomerates and loaded onto a multi-well plate with Mylar polymer film to support the sample. The multi-well plate was then placed into the diffractometer and analyzed using Cu K radiation (α1λ=1.54060 Å; α2=1.54443 Å; β=1.39225 Å; α1:α2 ratio=0.5) running in transmission mode (step size 0.0130° 2θ, step time 18.87s) using 40 kV/40 mA generator settings. Data were visualized and images generated using the HighScore Plus 4.7 desktop application (PANalytical, 2017).

Polarized Light Microscopy (PLM): The presence of crystallinity (birefringence) was determined using an Olympus BX53 microscope, equipped with cross-polarizing lenses and a Motic camera. Images were captured using Motic Images Plus 3.0. All images were recorded using the 20× objective, unless otherwise stated.

Thermogravimetric Analysis/Differential Scanning Calorimetry (TGA/DSC): Approximately, 5-10 mg of material was added into a pre-tared open aluminum pan and loaded into a TA Instruments Discovery SDT 650 Auto—Simultaneous DSC and held at room temperature. The sample was then heated at a rate of 10° C./min from 30° C. to 350° C. during which time the change in sample weight was recorded along with the heat flow response (DSC). Nitrogen was used as the sample purge gas, at a flow rate of 200 cm³/min.

Thermogravimetric/Differential Thermal Analysis (TG/DTA): Approximately, 5-10 mg of material was added into a pre-tared open aluminum pan and loaded into a TA Instruments Discovery SDT 650 Auto—Simultaneous DSC and held at room temperature. The sample was then heated at a rate of 10° C./min from 30° C. to 350° C. during which time the change in sample weight was recorded along with the heat flow response (DSC). Nitrogen was used as the sample purge gas, at a flow rate of 200 cm³/min.

Differential Scanning Calorimetry (DSC): Approximately, 1-5 mg of material was weighed into an aluminum DSC pan and sealed non-hermetically with an aluminum lid. The sample pan was then loaded into a TA Instruments Discovery DSC 2500 differential scanning calorimeter equipped with a RC90 cooler. The sample and reference were heated to melting at a scan rate of 10° C./min and the resulting heat flow response monitored. The sample was re-cooled to 20° C. and then reheated again to 220° C. all at 10° C./min. Nitrogen was used as the purge gas, at a flow rate of 50 cm³/min.

Infrared Spectroscopy (IR): Infrared spectroscopy was carried out on a Bruker ALPHA P spectrometer. Sufficient material was placed onto the center of the plate of the spectrometer and the spectra were obtained using the following parameters:
Resolution: 4 cm⁻¹
Background Scan Time: 16 scans Sample Scan Time: 16 scans
Data Collection: 4000 to 400 cm⁻¹
Result Spectrum: Transmittance
Software: OPUS version 6

Proton Nuclear Magnetic Resonance (¹H-NMR): ¹H-NMR experiments were performed on a Bruker AVIIIHD spectrometer equipped with a DCH cryoprobe operating at 500.12 MHz for protons. Experiments were performed in deuterated DMSO and each sample was prepared to ca. 10 mM concentration.

### Example 1: Synthesis and Characterization of Amorphous Compound 1 Mono-Tosylate

Synthesis of a batch amorphous Compound 1 mono-tosylate was carried out pursuant to Example 139 of U.S. Ser. No. 10/316,029, which as explained above, is hereby incorporated by reference in its entirety. Amorphous Compound 1 mono-tosylate was used as the starting material to produce the crystalline forms of Compound 1 mono-tosylate of the present disclosure.

XRPD Analysis: XRPD analysis of amorphous Compound 1 mono-tosylate was carried out on a PANalytical X'pert pro with PIXcel detector (128 channels), scanning the samples between 3 and 25° 2θ. The material was gently ground to release any agglomerates and loaded onto a multi-well plate with Mylar polymer film to support the sample. The multi-well plate was then placed into a diffractometer and analyzed using Cu K radiation (α₁, =1.54060 Å; α₂=1.54443 Å; β=1.39225 Å; α₁:α₂ ratio=0.05) running in transmission mode (step size 0.0130° 2θ, step time 18.87s) using 40 kV/40 mA generator settings. Data were visualized and images generated using the HighScore Plus 4.7 desktop application (PANalytical, 20917).

The resulting XRPD pattern for amorphous Compound 1 mono-tosylate was collected. A sharp signal was observed at low angles, possibly indicating a small amount of short-range order was in the sample. However, the pattern clearly shows the bulk of the material is amorphous.

TG/DSC Analysis of Amorphous Compound 1: The TG/DSC thermogram of amorphous Compound 1 was collected. The TG trace of amorphous Compound 1 displayed a 3.1 wt. % (1.11 equivalents of water) mass loss was observed from the onset of heating, likely due to surface moisture.

Additional mass losses were observed which appeared to be associated with decomposition.

The DSC trace showed a shallow endothermic event (onset at 184° C.) was observed, likely associated with the onset of decomposition, including the possible loss of toluene sulphonic acid.

PLM: Micrographs of amorphous Compound 1 mono-tosylate were made with non-polarized light and polarized light.

FT-IR Spectrum of Amorphous Compound 1: The FT-IR spectrum of amorphous Compound 1 was collected. It appears consistent with the structure of amorphous Compound 1. The signal at 1686 cm⁻¹ is likely due to the amide group of the molecule. Signals around 1650 cm⁻¹ to around 1560 cm⁻¹ are consistent with the C═N cyclic group and signals in the fingerprint region are consistent with toluenesulfonic acid.

Proton NMR Spectrum of Amorphous Compound 1 mono-tosylate: All of Compound 1 mono-tosylate's protons were accounted for in the ¹H NMR spectrum. The presence of the mono-tosylate was confirmed. Based upon the spectrum, it appears some residual ethanol and hexane solvents appeared to be present.

Results: Based upon the various analyses performed on amorphous Compound 1, which is the starting material for the preparation of crystalline forms of Compound 1 mono-tosylate, the starting material was indeed found to be amorphous. Particular properties of amorphous Compound 1 are set forth in Table 1 below.

TABLE 1
Summary of Properties of Amorphous Compound 1 Mono-Tosylate

| Analysis | Observations |
|---|---|
| XRPD | Amorphous |
| PLM | Glassy, plate-like particles. Did not appear birefringent |
| FT-IR | Signals characteristic with Compound 1 mono-tosylate |
| | functional groups present |
| NMR | Consistent with structure of Compound 1 mono-tosylate salt. |
| TG/DSC | Surface moisture loss observed below 100° C. Endothermic |
| | event associated with decomposition observed (onset 184° C.) |

### Example 2: Primary Polymorph Screen of Compound 1 Mono-Tosylate

24×50 mg (0.1 mmol) of amorphous Compound 1 mono-tosylate was weighed into 1.5 mL vials. Solvent was added to each vial in an attempt to prepare slurries. Post-solvent addition, slurries/solutions were thermally cycled (with agitation) between ambient temperature and 40° C. (4 h cycles at each condition) for 72 h.

Post-thermal cycling, where slurries were observed, solids were isolated via centrifugation filtration and the solids were analyzed by XRPD. The mother liquors were then evenly split into 3. Where solutions were observed post-thermal cycling, 1.2 mL anti-solvent was added to one of the 3 solutions and thermally cycled as before, but for 24 h. Any solids recovered post-thermal cycling were first isolated via centrifugation filtration before being analyzed by XRPD. If no solids were recovered, the solutions were stored in a fridge (at 5° C.) for 48 h.

The mother liquors were split into 3 for the following experiments:
(i) Evaporation at ambient temperature
(ii) Cooling to approximately 5° C. for 48 h
(iii) Subsequently cooled to approximately −18° C. if no solids were produced after 48 h at
(iv) 5° C.
(v) Anti-solvent addition at ambient temperature
(vi) If no solids were observed at ambient temperature (or post-thermal cycling, where applicable), the solutions were stored at 5° C. for 48 h and then 18° C. if no solids were produced after 48 h at 5° C.

TABLE 2
Solvents used in Primary Polymorph Screen
of Amorphous Compound 1 Mono-Tosylate

| | | Volume | |
|---|---|---|---|
| Solvent | ICH Class | Added (μL) | Anti-solvent |
| 1,4-Dioxane | 2 | 1200 | Heptane |
| 1-Butanol | 3 | 600 | Heptane |
| 2-Methyl THF | 3 | 1200 | Heptane |
| 2-Methyl-1-Propanol | 3 | 500 | Heptane |
| 2-Propanol | 3 | 500 | Heptane |
| Methanol/Water | 3 | 200 | Acetonitrile |
| (48:52% v/v) | | | |
| (calculated a_{w} 0.8) | | | |
| Ethanol/Water | 3 | 200 | Acetonitrile |
| (98.5:1.5% v/v) | | | |
| (calculated a_{w} 0.2) | | | |
| Acetone | 3 | 1200 | Heptane |
| Acetonitrile | 2 | 1200 | Isopropyl |
| | | | acetate |
| Anisole | 3 | 1200 | Heptane |
| Butyl Acetate | 3 | 1200 | Heptane |
| Dichloromethane | 2 | 1200 | Heptane |
| 1,4-Dioxane | 2 | 1200 | Heptane |
| Diisopropyl Ether | Unclassified | 1200 | Heptane |
| Dimethylcarbonate | Unclassified | 1200 | Heptane |
| Ethanol | 3 | 200 | Heptane |
| Ethyl Acetate | 3 | 1200 | Heptane |
| Isopropyl Acetate | 3 | 1200 | Heptane |
| Methanol | 2 | 200 | Heptane |
| Methylethyl Ketone | 3 | 1200 | Heptane |
| Methylisobutyl Ketone | 2 | 1200 | Heptane |
| N,N′-Dimethylacetamide | 2 | 200 | Heptane |
| Tetrahydrofuran | 2 | 1200 | Heptane |
| Toluene | 2 | 1200 | Heptane |
| Water | n/a | 400 | Acetonitrile |
| Diisopropyl Ether | Unclassified | 1200 | Heptane |

### Example 3: Post Thermal Cycling

Where slurries were observed from the samples of Example 1, solids from the slurries were isolated via centrifugation filtration, and the solids were analyzed by XRPD. The mother liquors were then evenly split into 3. Where solutions were observed post-thermal cycling, 1.2 ml anti-solvent was added to one of the 3 solutions and thermally cycled as before, but for 24 hours. Any solids recovered post-thermal cycling were first isolated via centrifugation filtration before being analyzed by XRPD. If no solids were recovered, the solutions were stored at 5° C. for 48 hours.

As explained above, the mother liquors were split into 3 for the following experiments:
(i) Evaporation at ambient temperature;
(ii) cooling to approximately 5° C. for 48 hours and if no solids were produced, subsequently cooling to approximately −18° C.; and
(iii) Anti-solvent addition at ambient temperature
(iv) If no solids were observed at ambient temp or post-thermal cycling, where applicable, the solutions were stored at 5° C. for 48 hours, and if no solids were produced, they were stored for 48 hours at 5° C.

Any new solids which produced patterns by XRPD were dried at 40° C. for 24 hours before being analyzed again. Potential New Patterns were also analyzed by ¹H NMR, PLM and TG/DSC.

Observations made during the thermal cycling experiments with Compound 1 mono-tosylate solutions in different solvent systems are set forth in Table 3 below.

TABLE 3

| | | Volume | Post-Solvent | |
|---|---|---|---|---|
| | Solvent System | Added (mL) | Addition | Post-Thermal Cycling |
| 1 | 1,4-Dioxane | 1.2 | Slurry | Thick white slurry |
| 2 | 1-Butanol | 0.6 | Solution | Slightly yellow solution |
| 3 | 2-Methyl THF | 1.2 | Slurry | Thick white slurry |
| 4 | 2-Methyl-1-Propanol | 0.5 | Slurry | Slightly yellow solution |
| 5 | 2-Propanol | 0.5 | Slurry | Slightly yellow solution |
| 6 | Methanol/Water (48:52% v/v) (calculated a_{w} 0.8) | 0.2 | Solution | Slightly yellow solution |
| 7 | Ethanol/Water (98.5:1.5% v/v) (calculated a_{w} 0.2) | 0.2 | Solution | Slightly yellow solution |
| 8 | Acetone | 1.2 | Slurry | Deep orange solution |
| 9 | Acetonitrile | 1.2 | Slurry | Off-white solid adhered to the vial, colourless solution |
| 10 | Anisole | 1.2 | Slurry | Thin, off-white slurry |
| 11 | Butyl Acetate | 1.2 | Slurry | Off-white slurry |
| 12 | Dichloromethane | 1.2 | Slurry | Off-white slurry. Spherical particles |
| 13 | Diisopropyl Ether | 1.2 | Slurry | Off-white slurry |
| 14 | Dimethylcarbonate | 1.2 | Slurry | Off-white slurry |
| 15 | Ethanol | | Solution | Slightly yellow solution |
| 16 | Ethyl Acetate | 1.2 | Slurry | Off-white slurry |
| 17 | Isopropyl Acetate | 1.2 | Slurry | Off-white slurry |
| 18 | Methanol | 0.2 | Solution | Slightly yellow solution |
| 19 | Methylethyl Ketone | 1.2 | Slurry | Orange solid adhered to the vial, pink solution |
| 20 | Methylisobutyl Ketone | 1.2 | Slurry | Off-white slurry |
| 21 | N,N′-Dimethylacetamide | 0.2 | Solution | Slightly yellow solution |
| 22 | Tetrahydrofuran | 1.2 | Slurry | Off-white slurry |
| 23 | Toluene | 1.2 | Slurry | Off-white slurry |
| 24 | Water | 0.4 | Slurry | Some colorless gum in solution |

| | Anti-Solvent Added | | | |
|---|---|---|---|---|
| | and Thermal Cycling | Anti-Solvent | Post-Anti- | |
| | Repeated? | Added (1.2 mL) | Solvent Addition | Post-Repeat Thermal Cycling |
| 1 | N | | | |
| 2 | Y | Heptane | Slurry | Off-white slurry |
| 3 | N | | | |
| 4 | Y | Heptane | Slurry | Off-white slurry |
| 5 | Y | Heptane | Slurry | Off-white slurry |
| 6 | Y | Acetonitrile | Slurry | Off-white tacky material adhered to the vial, colourless solution |
| 7 | Y | Acetonitrile | Slurry | Off-white tacky material adhered to the vial, colourless solution |
| 8 | Y | Heptane | Slurry | Brown tacky material adhered to the vial, colourless solution |
| 9 | N | | | |
| 10 | N | | | |
| 11 | N | | | |
| 12 | N | | | |
| 13 | N | | | |
| 14 | N | | | |
| 15 | Y | Heptane | Thin slurry | Off-white tacky material adhered to the vial, colourless solution |
| 16 | N | | | |
| 17 | N | | | |
| 18 | Y | Heptane | Solution | Off-white tacky material adhered to the vial, colourless solution |
| 19 | N | | | |
| 20 | N | | | |
| 21 | Y | Acetonitrile | Slurry | Off-white tacky material adhered to the vial, colourless solution |
| 22 | N | | | |
| 23 | N | | | |
| 24 | Y | Acetonitrile | Solution | Colourless solution |

The results of the primary polymorph screen are set forth in Table 4 and Table 5 below.

TABLE 4

| | | Post-Thermal Cycling | | | Cooling | | Anti-Solvent |
|---|---|---|---|---|---|---|---|
| No. | Solvent System | Wet | Dry | Evaporation | 5° C. | −18° C. | Addition |
| 1 | 1,4-Dioxane | P2 | +, P2 | NS | NS | NS | NS |
| 2 | 1-Butanol | AS, AMPH | AMPH | ▪ | NS | NS | TC, AMPH |
| 3 | 2-Methyl THF | P1 | S, P1 | NS | NS | NS | NS |
| 4 | 2-Methyl-1-Propanol | AS, AMPH | AMPH | ▪ | NS | NS | TC, AMPH |
| 5 | 2-Propanol | AS, AMPH | AMPH | ▪ | NS | ◯ | TC, AMPH |
| 6 | Methanol/Water (48:52 | AS, ▪ | ▪ | AMPH | NS | NS | ▪ |
| | % v/v) (calculated a_{w} 0.8) | | | | | | |
| 7 | Ethanol/Water (98.5:1.5 | AS, ▪ | ▪ | AMPH | NS | NS | C, *, AMPH |
| | % v/v) (calculated a_{w} 0.2) | | | | | | |
| 8 | Acetone | AS, ▪ | ▪ | ▪ | NS | ◯ | ▪ |
| 9 | Acetonitrile | *, AMPH | *, AMPH | NS | NS | NS | NS |
| 10 | Anisole | AMPH | AMPH | NS | NS | NS | NS |
| 11 | Butyl Acetate | AMPH | AMPH | NS | NS | NS | NS |
| 12 | Dichloromethane | P1 | +, P1 | NS | NS | NS | NS |
| 13 | Diisopropyl Ether | AMPH | AMPH | NS | NS | NS | NS |
| 14 | Dimethylcarbonate | AMPH | AMPH | NS | NS | ◯ | NS |
| 15 | Ethanol | ▪ | ▪ | AMPH | NS | NS | ▪ |
| 16 | Ethyl Acetate | AMPH | AMPH | NS | NS | ◯ | NS |
| 17 | Isopropyl Acetate | AMPH | AMPH | NS | NS | NS | NS |
| 18 | Methanol | ▪ | ▪ | AMPH | NS | NS | ▪ |
| 19 | Methylethyl Ketone | AMPH | P1 | NS | NS | ◯ | NS |
| 20 | Methylisobutyl Ketone | AMPH | AMPH | *, AMPH | NS | ◯ | NS |
| 21 | N,N′-Dimethylacetamide | ▪ | ▪ | NS | NS | NS | ▪ |
| 22 | Tetrahydrofuran | P3 | P4 | NS | NS | NS | NS |
| 23 | Toluene | AMPH | AMPH | NS | NS | NS | NS |
| 24 | Water | NS | NS | NS | NS | NS | NS |

TABLE 5
The key for analyzing Table 4.

| | | | | | | Tacky | |
|---|---|---|---|---|---|---|---|
| | | | | | | material | Very |
| | | | | | | adhered to | little |
| | | | | | | vial - no | solid - no |
| | | | | | | XRPD | XRPD |
| Pattern 1 | Pattern 2 | Pattern 3 | Pattern 4 | Amorphous | No solid | collected | collected |
| P1 | P2 | P3 | P4 | AMPH | NS | ▪ | ◯ |

| | Some | | Some | | | |
|---|---|---|---|---|---|---|
| Poor | weak | Additional | signal | Anti-solvent | Thermally | Cooled to |
| crystallinity | signals | signals | shifting | added | cycled | 5° C. |
| PC | * | + | S | AS | TC | C |

### Example 4: Secondary Polymorph Screen—Formation of Crystalline Forms 1 and 4

To prepare crystalline forms 1 and 4 of Compound 1 mono-tosylate, 2×500 mg (1.01 mmol) of amorphous Compound 1 mono-tosylate was placed into 20 ml vials. To each of these vials, 12 ml of solvent (dichloromethane for crystalline form 1 and tetrahydrofuran for crystalline form 4) was added to prepare slurries. Volumes/mass used were a direct scale-up from the primary screen discussed above. The slurries were thermally cycled (with agitation) between ambient temperature and 40° C. (4-hour cycles at each condition) for 72 hours. Post-thermal cycling, an aliquot of solid was removed from each vial using a micro-spatula and analyzed by XRPD to confirm that the desired form was produced. The remainder of each slurry was then filtered by Buchner filtration. The solids were dried on the filter bed for no longer than 1 minute before being transferred to pre-weighed 20 ml vials. The solids were then dried under vacuum at ambient temperature for 2 hours. Another aliquot of each solid was analyzed by XRPD post-drying. Crystalline form 4 was expected to be produced post-drying (conversion from crystalline form 3) but crystalline form 3 remained also. As such, the solids were transferred to an oven at 40° C. (ambient pressure) and subsequently dried for approximately 18 hours. Analysis of a further aliquot of solid confirmed that all crystalline form 3 produced had converted to crystalline form 4. Crystalline form 1 yield: 86.1%. Crystalline form 4 yield: 84.2%.

### Example 5: Formation of Crystalline Form 5

Crystalline form 5 of Compound 1 mono-tosylate was obtained during a 7 day stability study for crystalline forms 1 and 4. In the study, 3×20 mg (0.04 mmol) of crystalline forms 1 and 4, and amorphous Compound 1 mono-tosylate, were individually weighted into 1.5 mL vials and stored under the following conditions for 7 days:
(i) 40° C./75% relative humidity (RH) (open vial);
(ii) 80° C./ambient humidity (sealed vial); and
(iii) Ambient conditions (open vial).

After 7 days, any remaining solids recovered were analyzed by XRPD to assess form purity and HPLC changes in chemical purity. The analysis showed that crystalline form 4 had converted into a new crystalline form: crystalline form 5. More crystalline form 5 was prepared by storing 100 mg of crystalline form 4 at for C/75% RH for approximately 4 days.

### Example 6: Polymorph Stability Study (Competitive Slurrying and Preparation of Crystalline Form 6)

To prepare solutions saturated with Compound 1 mono-tosylate for competitive slurrying, 2×100 mg (0.20 mmol) amorphous Compound 1 mono-tosylate was weighed into 2×4 mL vials. To each vial, 500 μL of ethanol was added and the vials were manually shaken for a few seconds. Complete dissolution was noted. A further about 20 mg of amorphous Compound 1 mono-tosylate was added to each vial and the vials were agitated for about 3 hours: one vial at ambient temperature and one vial at 60° C. After about 3 hours, both were solutions. More amorphous Compound 1 mono-tosylate (about 30 mg) was added to each vial and they were shaken at their respective temperatures again for about another hour. Both appeared to be slurries. To remove the excess solids, the slurries were filtered centrifugally and the mother liquors each placed into separate, clean vials. To each vial containing saturated mother liquor, 10 mg of crystalline form 1, crystalline form 4 and crystalline form 5 (total of 30 mg of solid added to each vial) was added and slurries were produced. The slurries were then agitated for 48 hours: one vial at ambient temperature and one vial at 60° C. After 48 hours, the solids were recovered via centrifugal filtration and analyzed by XRPD. The XRPD plate containing the solids was then dried at 40° C. for 2 hours and re-analyzed. New crystalline form 6 was observed by XPRD (and was retained on drying).

### Example 7: Patterns 1, 4, 5 and 6

To prepare a solution saturated with amorphous Compound 1 mono-tosylate for competitive slurrying, 50 mg (0.10 mmol) of amorphous Compound 1 mono-tosylate was weighed into a 4 mL vial. To the vial, 1 mL of 2-propanol was added, and the slurry was agitated for about 4 hours. A further about 30 to about 40 mg of amorphous Compound 1 mono-tosylate was added to the vial after 30 min as all originally added amorphous Compound 1 mono-tosylate had dissolved. To remove the excess solids, the slurry was filtered centrifugally, and the mother liquor was placed into a clean vial. To the vial containing the saturated mother liquor, 10 mg of crystalline form 1, crystalline form 4, crystalline form 5 and crystalline form 6 (total of 40 mg of solid added) was added and a slurry was produced. The slurry was agitated for 72 hours at ambient temperature. After 72 hours, the solids were recovered via centrifugal filtration and analyzed by XRPD.

### Example 8: Pattern 6 Re-Preparation

50 mg (0.51 mmol) of amorphous Compound 1 mono-tosylate was weighed into a 4 mL vial. 1 mL ethanol was added, and a very slightly turbid solution resulted after manual shaking for a few seconds. To this solution, 5% (12.5 mg) crystalline form 6 seed (recovered as discussed above) was added and the slurry was agitated at ambient temperature for 24 hours. After 24 hours, a significant amount of off-white precipitate was observed. The solid was isolated via centrifugal filtration and an aliquot of wet solid was analyzed by XRPD. Crystalline form 6 was confirmed. The remainder of the isolated solid was added to a pre-weighed vial and dried in an oven at 40° C. for approximately 5 hours. Yield: 64.7%.

### Example 9: Analyses Performed on Compound 1 Mono-Tosylate Crystalline Forms

### XRPD Analysis

XRPD Analyses were performed on Compound 1 mono-tosylate crystalline forms 1, 2, 4, 5 and 6 of the present disclosure as set forth above.

Compound 1 mono-tosylate crystalline form 1 of the present disclosure prepared pursuant to the previous examples was subjected to an XRPD analysis as described herein. The XRPD is set forth in FIG. 1. Particular signals of interest are set forth in Table 6 below.

TABLE 6
XRPD Diffraction Signals For Compound 1 mono-tosylate
Crystalline Form 1 (°2θ ± 0.2).

| No. | Pos. [°2θ] | d-spacing [Å] | Height [cts] | Rel. Int. [%] |
|---|---|---|---|---|
| 1 | 4.0 | 22.1 | 77.4 | 8.1 |
| 2 | 4.4 | 20.0 | 74.7 | 7.8 |
| 3 | 5.2 | 17.0 | 742.2 | 77.2 |
| 4 | 6.1 | 14.5 | 961.1 | 100.0 |
| 5 | 8.4 | 10.6 | 61.5 | 6.4 |
| 6 | 9.9 | 9.0 | 24.5 | 2.6 |
| 7 | 10.3 | 8.6 | 56.1 | 5.8 |
| 8 | 10.8 | 8.2 | 22.7 | 2.4 |
| 9 | 12.2 | 7.3 | 75.6 | 7.9 |
| 10 | 13.8 | 6.4 | 22.3 | 2.3 |
| 11 | 15.6 | 5.7 | 441.9 | 46.0 |
| 12 | 15.8 | 5.6 | 575.1 | 59.8 |
| 13 | 16.1 | 5.5 | 216.2 | 22.5 |
| 14 | 16.5 | 5.4 | 152.9 | 15.9 |
| 15 | 16.7 | 5.3 | 108.5 | 11.3 |
| 16 | 17.2 | 5.1 | 222.4 | 23.1 |
| 17 | 17.8 | 5.0 | 276.9 | 28.8 |
| 18 | 18.2 | 4.9 | 252.7 | 26.3 |
| 19 | 18.8 | 4.7 | 296.1 | 30.8 |
| 20 | 19.1 | 4.6 | 233.4 | 24.3 |
| 21 | 19.5 | 4.6 | 259.5 | 27.0 |
| 22 | 20.7 | 4.3 | 268.9 | 28.0 |
| 23 | 22.3 | 4.0 | 274.4 | 28.6 |
| 24 | 22.8 | 3.9 | 195.8 | 20.4 |
| 25 | 23.1 | 3.9 | 184.3 | 19.2 |
| 26 | 23.5 | 3.8 | 213.2 | 22.2 |
| 27 | 23.9 | 3.7 | 283.0 | 29.5 |
| 28 | 24.6 | 3.6 | 148.7 | 15.5 |
| 29 | 25.1 | 3.5 | 157.2 | 16.4 |
| 30 | 26.0 | 3.4 | 157.7 | 16.4 |
| 31 | 26.5 | 3.4 | 166.2 | 17.3 |
| 32 | 27.0 | 3.3 | 233.1 | 24.3 |
| 33 | 27.6 | 3.2 | 106.3 | 11.1 |
| 34 | 28.8 | 3.1 | 103.4 | 10.8 |
| 35 | 30.2 | 3.0 | 65.3 | 6.8 |
| 36 | 31.2 | 2.9 | 43.8 | 4.6 |
| 37 | 32.3 | 2.8 | 52.1 | 5.4 |
| 38 | 33.5 | 2.7 | 27.2 | 2.8 |
| 39 | 34.4 | 2.6 | 31.0 | 3.2 |

The Compound 1 mono-tosylate crystalline form 2 of the present disclosure was prepared as described above, and was subjected to XRPD analysis. The XRPD for crystalline form 2 is set forth in FIG. 4, and the signals are set forth in Table 7.

TABLE 7
XRPD Diffraction Signals For Compound 1 Mono-Tosylate
Crystalline Form 2 (°2θ ± 0.2).

| No. | Pos. [°2θ] | d-spacing [Å] | Height [cts] | Rel. Int. [%] |
|---|---|---|---|---|
| 1 | 5.0 | 17.8 | 1327.6 | 100.0 |
| 2 | 5.4 | 16.5 | 264.5 | 19.9 |
| 3 | 5.9 | 14.9 | 449.5 | 33.9 |
| 4 | 7.2 | 12.2 | 153.4 | 11.6 |
| 5 | 8.2 | 10.8 | 67.7 | 5.1 |
| 6 | 9.9 | 8.9 | 66.8 | 5.0 |
| 7 | 10.8 | 8.2 | 62.5 | 4.7 |
| 8 | 12.1 | 7.3 | 28.2 | 2.1 |
| 9 | 15.0 | 5.9 | 190.1 | 14.3 |
| 10 | 15.5 | 5.7 | 686.7 | 51.7 |
| 11 | 16.4 | 5.4 | 345.3 | 26.0 |
| 12 | 16.8 | 5.3 | 82.5 | 6.2 |
| 13 | 17.3 | 5.1 | 251.5 | 18.9 |
| 14 | 17.6 | 5.0 | 130.8 | 9.9 |
| 15 | 18.2 | 4.9 | 240.9 | 18.2 |
| 16 | 18.7 | 4.8 | 269.9 | 20.3 |
| 17 | 19.6 | 4.5 | 264.9 | 20.0 |
| 18 | 20.4 | 4.4 | 161.8 | 12.2 |
| 19 | 20.8 | 4.3 | 235.4 | 17.7 |
| 20 | 21.6 | 4.1 | 263.4 | 19.8 |
| 21 | 21.9 | 4.1 | 218.5 | 16.5 |
| 22 | 22.4 | 4.0 | 119.9 | 9.0 |
| 23 | 22.9 | 3.9 | 133.8 | 10.1 |
| 24 | 23.6 | 3.8 | 221.3 | 16.7 |
| 25 | 24.4 | 3.6 | 37.4 | 2.8 |
| 26 | 25.1 | 3.6 | 51.9 | 3.9 |
| 27 | 26.5 | 3.4 | 126.4 | 9.5 |
| 28 | 27.7 | 3.2 | 66.9 | 5.0 |
| 29 | 29.2 | 3.1 | 97.9 | 7.4 |
| 30 | 30.9 | 2.9 | 25.4 | 1.9 |
| 31 | 33.1 | 2.7 | 39.2 | 3.0 |

The Compound 1 mono-tosylate crystalline form 3 of the present disclosure was prepared as described above, and was subjected to XRPD analysis. The XRPD for crystalline form 3 is set forth in FIG. 18, and the signals are set forth in Table 8.

TABLE 8
XRPD Diffraction Signals For Compound 1 Mono-Tosylate
Crystalline Form 3 (°2θ ± 0.2).

| No. | Pos. [°2θ] | d-spacing [Å] | Height [cts] | Rel. Int. [%] |
|---|---|---|---|---|
| 1 | 3.3 | 26.5 | 92.2 | 6.0 |
| 2 | 5.0 | 17.6 | 1528.2 | 100.0 |
| 3 | 5.6 | 15.9 | 645.9 | 42.3 |
| 4 | 6.3 | 13.9 | 442.3 | 28.9 |
| 5 | 6.8 | 13.1 | 344.4 | 22.5 |
| 6 | 6.9 | 12.9 | 247.0 | 16.2 |
| 7 | 7.5 | 11.8 | 49.3 | 3.2 |
| 8 | 9.0 | 9.9 | 39.6 | 2.6 |
| 9 | 9.8 | 9.0 | 47.9 | 3.1 |
| 10 | 10.3 | 8.6 | 110.9 | 7.3 |
| 11 | 12.3 | 7.2 | 91.9 | 6.0 |
| 12 | 13.5 | 6.6 | 65.9 | 4.3 |
| 13 | 14.8 | 6.0 | 34.5 | 2.3 |
| 14 | 15.6 | 5.7 | 1528.5 | 100.0 |
| 15 | 16.1 | 5.5 | 319.4 | 20.9 |
| 16 | 16.7 | 5.3 | 312.8 | 20.5 |
| 17 | 17.0 | 5.2 | 279.6 | 18.3 |
| 18 | 18.1 | 4.9 | 631.5 | 41.3 |
| 19 | 18.3 | 4.9 | 600.2 | 39.3 |
| 20 | 18.9 | 4.7 | 575.0 | 37.6 |
| 21 | 19.2 | 4.6 | 287.1 | 18.8 |
| 22 | 20.6 | 4.3 | 136.5 | 8.9 |
| 23 | 20.8 | 4.3 | 129.7 | 8.5 |
| 24 | 21.8 | 4.1 | 731.9 | 47.9 |
| 25 | 22.4 | 4.0 | 825.4 | 54.0 |
| 26 | 23.6 | 3.8 | 135.2 | 8.8 |
| 27 | 24.1 | 3.7 | 189.5 | 12.4 |
| 28 | 24.8 | 3.6 | 74.2 | 4.9 |
| 29 | 25.3 | 3.5 | 376.3 | 24.6 |
| 30 | 25.5 | 3.5 | 376.0 | 24.6 |
| 31 | 26.1 | 3.4 | 76.4 | 5.0 |
| 32 | 26.8 | 3.3 | 182.8 | 12.0 |
| 33 | 27.1 | 3.3 | 178.3 | 11.7 |
| 34 | 29.1 | 3.1 | 110.3 | 7.2 |
| 35 | 29.6 | 3.0 | 92.0 | 6.0 |
| 36 | 30.9 | 2.9 | 69.2 | 4.5 |
| 37 | 32.5 | 2.8 | 68.1 | 4.5 |

The Compound 1 mono-tosylate crystalline form 4 of the present disclosure was prepared as described above, and was subjected to XRPD analysis. The XRPD for crystalline form 4 is set forth in FIG. 7, and the signals are set forth in Table 9.

TABLE 9
XRPD Diffraction Signals For Compound 1 Mono-Tosylate
Crystalline Form 4 (°2θ ± 0.2).

| No. | Pos. [°2θ] | d-spacing [Å] | Height [cts] | Rel. Int. [%] |
|---|---|---|---|---|
| 1 | 3.2 | 27.4 | 93.4 | 12.5 |
| 2 | 4.1 | 21.5 | 96.7 | 13.0 |
| 3 | 5.0 | 17.7 | 746.1 | 100.0 |
| 4 | 5.6 | 15.8 | 389.7 | 52.2 |
| 5 | 6.0 | 14.7 | 237.0 | 31.8 |
| 6 | 6.3 | 13.9 | 254.2 | 34.1 |
| 7 | 6.8 | 13.0 | 173.1 | 23.2 |
| 8 | 7.9 | 11.1 | 20.3 | 2.7 |
| 9 | 8.9 | 10.0 | 16.9 | 2.3 |
| 10 | 10.1 | 8.7 | 19.8 | 2.7 |
| 11 | 12.4 | 7.1 | 35.5 | 4.8 |
| 12 | 13.4 | 6.6 | 18.9 | 2.5 |
| 13 | 13.8 | 6.4 | 25.3 | 3.4 |
| 14 | 15.6 | 5.7 | 682.1 | 91.4 |
| 15 | 16.0 | 5.5 | 208.0 | 27.9 |
| 16 | 16.7 | 5.3 | 237.6 | 31.9 |
| 17 | 17.0 | 5.2 | 252.0 | 33.8 |
| 18 | 17.3 | 5.1 | 220.0 | 29.5 |
| 19 | 17.7 | 5.0 | 282.9 | 37.9 |
| 20 | 18.0 | 4.9 | 377.3 | 50.6 |
| 21 | 18.9 | 4.7 | 387.3 | 51.9 |
| 22 | 19.4 | 4.6 | 219.2 | 29.4 |
| 23 | 19.7 | 4.5 | 158.9 | 21.3 |
| 24 | 20.9 | 4.2 | 160.5 | 21.5 |
| 25 | 21.7 | 4.1 | 422.9 | 56.7 |
| 26 | 22.3 | 4.0 | 399.0 | 53.5 |
| 27 | 22.9 | 3.9 | 238.4 | 32.0 |
| 28 | 24.1 | 3.7 | 161.2 | 21.6 |
| 29 | 25.2 | 3.5 | 187.9 | 25.2 |
| 30 | 25.5 | 3.5 | 192.8 | 25.9 |
| 31 | 27.0 | 3.3 | 120.9 | 16.2 |
| 32 | 27.8 | 3.2 | 64.8 | 8.7 |
| 33 | 28.2 | 3.2 | 57.8 | 7.8 |
| 34 | 29.0 | 3.1 | 72.9 | 9.8 |
| 35 | 30.8 | 2.9 | 30.2 | 4.1 |

The Compound 1 mono-tosylate crystalline form 5 of the present disclosure was prepared as described above, and was subjected to XRPD analysis as discussed above. The XRPD for crystalline form 5 is set forth in FIG. 10. The signals for the XRPD are set forth in Table 10.

TABLE 10
XRPD Diffraction Signals For Compound 1 Mono-Tosylate
Crystalline Form 5 (°2θ ± 0.2).

| No. | Pos. [°2θ] | d-spacing [Å] | Height [cts] | Rel. Int. [%] |
|---|---|---|---|---|
| 1 | 3.3 | 27.0 | 419.0 | 20.1 |
| 2 | 6.2 | 14.2 | 1629.7 | 78.2 |
| 3 | 6.6 | 13.5 | 626.1 | 30.0 |
| 4 | 7.3 | 12.1 | 730.2 | 35.0 |
| 5 | 8.0 | 11.1 | 54.6 | 2.6 |
| 6 | 9.5 | 9.3 | 80.2 | 3.9 |
| 7 | 10.6 | 8.4 | 370.5 | 17.8 |
| 8 | 11.9 | 7.4 | 159.7 | 7.7 |
| 9 | 12.4 | 7.1 | 443.2 | 21.3 |
| 10 | 14.0 | 6.3 | 200.3 | 9.6 |
| 11 | 14.6 | 6.1 | 665.4 | 31.9 |
| 12 | 15.4 | 5.8 | 1004.0 | 48.2 |
| 13 | 15.9 | 5.6 | 2084.6 | 100.0 |
| 14 | 16.2 | 5.5 | 605.2 | 29.0 |
| 15 | 16.7 | 5.3 | 944.6 | 45.3 |
| 16 | 16.7 | 5.3 | 913.4 | 43.8 |
| 17 | 17.1 | 5.2 | 372.3 | 17.9 |
| 18 | 17.2 | 5.1 | 397.6 | 19.1 |
| 19 | 17.6 | 5.0 | 297.1 | 14.3 |
| 20 | 17.9 | 5.0 | 564.5 | 27.1 |
| 21 | 18.2 | 4.9 | 456.0 | 21.9 |
| 22 | 18.6 | 4.8 | 358.5 | 17.2 |
| 23 | 18.9 | 4.7 | 852.6 | 40.9 |
| 24 | 19.1 | 4.6 | 1006.5 | 48.3 |
| 25 | 19.8 | 4.5 | 696.5 | 33.4 |
| 26 | 20.2 | 4.4 | 424.3 | 20.4 |
| 27 | 20.8 | 4.3 | 617.2 | 29.6 |
| 28 | 21.2 | 4.2 | 580.2 | 27.8 |
| 29 | 21.7 | 4.1 | 239.2 | 11.5 |
| 30 | 22.1 | 4.0 | 229.9 | 11.0 |
| 31 | 22.4 | 4.0 | 299.5 | 14.4 |
| 32 | 22.6 | 3.9 | 371.6 | 17.8 |
| 33 | 22.9 | 3.9 | 1277.2 | 61.3 |
| 34 | 23.4 | 3.8 | 1151.5 | 55.2 |
| 35 | 23.6 | 3.8 | 1547.3 | 74.2 |
| 36 | 24.2 | 3.7 | 239.2 | 11.5 |
| 37 | 24.8 | 3.6 | 805.2 | 38.6 |
| 38 | 25.4 | 3.5 | 357.0 | 17.1 |
| 39 | 25.7 | 3.5 | 404.6 | 19.4 |
| 40 | 26.0 | 3.4 | 254.0 | 12.2 |
| 41 | 26.5 | 3.4 | 401.6 | 19.3 |
| 42 | 27.0 | 3.3 | 374.7 | 18.0 |
| 43 | 27.4 | 3.3 | 227.3 | 10.9 |
| 44 | 27.9 | 3.2 | 619.6 | 29.7 |
| 45 | 28.4 | 3.1 | 363.3 | 17.4 |
| 46 | 28.8 | 3.1 | 210.3 | 10.1 |
| 47 | 29.4 | 3.0 | 258.7 | 12.4 |
| 48 | 30.1 | 3.0 | 184.5 | 8.9 |
| 49 | 30.4 | 2.9 | 145.6 | 7.0 |
| 50 | 31.2 | 2.9 | 85.1 | 4.1 |
| 51 | 31.6 | 2.8 | 64.2 | 3.1 |
| 52 | 32.1 | 2.8 | 44.3 | 2.1 |
| 53 | 33.3 | 2.7 | 146.8 | 7.0 |

The Compound 1 mono-tosylate crystalline form 6 of the present disclosure was prepared as described above, and was subjected to XRPD analysis as discussed above. The XRPD for crystalline form 6 is set forth in FIG. 12. The signals for the XRPD are set forth in Table 11.

TABLE 11
XRPD Diffraction Signals For Compound 1 Mono-Tosylate
Crystalline Form 6 (°2θ ± 0.2).

| No. | Pos. [°2θ] | d-spacing [Å] | Height [cts] | Rel. Int. [%] |
|---|---|---|---|---|
| 1 | 8.0 | 11.0 | 561.5 | 12.5 |
| 2 | 8.4 | 10.6 | 268.9 | 6.0 |
| 3 | 9.7 | 9.1 | 820.5 | 18.3 |
| 4 | 10.9 | 8.1 | 671.5 | 15.0 |
| 5 | 11.2 | 7.9 | 1676.0 | 37.4 |
| 6 | 12.0 | 7.4 | 164.5 | 3.7 |
| 7 | 12.8 | 6.9 | 723.6 | 16.1 |
| 8 | 13.5 | 6.5 | 723.3 | 16.1 |
| 9 | 13.9 | 6.4 | 410.3 | 9.2 |
| 10 | 14.5 | 6.1 | 1190.0 | 26.5 |
| 11 | 15.0 | 5.9 | 150.3 | 3.4 |
| 12 | 15.4 | 5.7 | 408.4 | 9.1 |
| 13 | 15.7 | 5.7 | 472.4 | 10.5 |
| 14 | 16.1 | 5.5 | 1035.5 | 23.1 |
| 15 | 16.8 | 5.3 | 469.7 | 10.5 |
| 16 | 17.1 | 5.2 | 232.5 | 5.2 |
| 17 | 17.6 | 5.1 | 1355.4 | 30.2 |
| 18 | 18.3 | 4.9 | 4485.7 | 100.0 |
| 19 | 18.7 | 4.8 | 323.5 | 7.2 |
| 20 | 19.1 | 4.6 | 283.5 | 6.3 |
| 21 | 19.6 | 4.5 | 666.7 | 14.9 |
| 22 | 20.0 | 4.4 | 769.6 | 17.2 |
| 23 | 20.4 | 4.4 | 768.3 | 17.1 |
| 24 | 20.9 | 4.3 | 1617.6 | 36.1 |
| 25 | 21.5 | 4.1 | 583.9 | 13.0 |
| 26 | 21.9 | 4.1 | 570.9 | 12.7 |
| 27 | 22.2 | 4.0 | 930.2 | 20.7 |
| 28 | 22.5 | 4.0 | 527.3 | 11.8 |
| 29 | 23.0 | 3.9 | 1046.8 | 23.3 |
| 30 | 23.3 | 3.8 | 479.0 | 10.7 |
| 31 | 23.5 | 3.8 | 439.2 | 9.8 |
| 32 | 24.0 | 3.7 | 525.1 | 11.7 |
| 33 | 24.4 | 3.7 | 1264.4 | 28.2 |
| 34 | 24.6 | 3.6 | 1211.8 | 27.0 |
| 35 | 25.1 | 3.5 | 350.8 | 7.8 |
| 36 | 25.6 | 3.5 | 736.0 | 16.4 |
| 37 | 26.2 | 3.4 | 493.9 | 11.0 |
| 38 | 26.9 | 3.3 | 362.9 | 8.1 |
| 39 | 27.2 | 3.3 | 344.8 | 7.7 |
| 40 | 27.7 | 3.2 | 393.6 | 8.8 |
| 41 | 28.0 | 3.2 | 269.0 | 6.0 |
| 42 | 28.5 | 3.1 | 266.8 | 6.0 |
| 43 | 29.2 | 3.1 | 763.7 | 17.0 |
| 44 | 29.6 | 3.0 | 449.9 | 10.0 |
| 45 | 30.7 | 2.9 | 122.9 | 2.7 |
| 46 | 31.1 | 2.9 | 233.6 | 5.2 |
| 47 | 31.7 | 2.8 | 72.0 | 1.6 |
| 48 | 32.2 | 2.8 | 92.3 | 2.1 |
| 49 | 33.0 | 2.7 | 204.0 | 4.6 |
| 50 | 33.7 | 2.7 | 87.2 | 1.9 |
| 51 | 34.1 | 2.6 | 195.9 | 4.4 |
| 52 | 34.5 | 2.6 | 177.0 | 4.0 |

In some embodiments, the Compound 1 mono-tosylate crystalline forms described herein comprise one or more corresponding XRPD signals selected from Tables 6-11. In some embodiments, the one or more XRPD signals selected from Tables 6-11 are signals having a relative intensity of at least 10%, at least 20%, or at least 30%. In some embodiments, the Compound 1 mono-tosylate crystalline form comprises the XRPD signals selected from corresponding Tables 6-11 (i.e. form 1 and Table 6, form 2 and Table 7, form 3 and Table 8, form 4 and Table 9, form 5 and Table 10, or form 6 and Table 11) having a relative intensity of at least 10%. In some embodiments, the relative intensity is at least 20%. In some embodiments, the relative intensity is at least 25%. In some embodiments, the relative intensity is at least 30%. In some embodiments, the relative intensity is at least 50%. In some embodiments, the relative intensity is at least 90%. In some embodiments, the relative intensity is 100%.

The Compound 1 mono-tosylate crystalline forms of the present disclosure were prepared as described above, and subjected to TG/DSC analysis as discussed above. The signals for the TG/DSC traces are set forth in Table 12.

TABLE 12
TG/DSC Signals For Compound 1 Mono-Tosylate
Crystalline Forms 1-2 and 4-6.

| Compound 1 | Onset | Signal | |
|---|---|---|---|
| mono-tosylate | Temperature | Temperature | Enthalpy |
| crystalline form | (° C.) | (° C.) | J/g |
| Amorphous | 184.0 | 202.1 | 94.1 |
| Form 1 | 35.7 | 52.9 | 21.9 |
| Form 1 | 112.7 | 128.9 | 32.9 |
| Form 1 | 188.4 | 201.3 | 0.7 |
| Form 2 | 122.6 | 131.0 | 21.1 |
| Form 2 | 184.1 | 199.7 | 56.5 |
| Form 4 | 101.5 | 115.3 | 148.3 |
| Form 4 | 182.0 | 199.9 | 126.6 |
| Form 5 | 146.4 | N/A | N/A |
| Form 5 | 182.9 | 186.8, 198.7 | 99.8 |
| Form 5 | 218.5 | 223.8 | −6.9 |
| Form 6 | 196.2 | 201.5 | 165.0 |

In some embodiments, the Compound 1 mono-tosylate crystalline forms described herein comprise one, two, or three corresponding TG/DSC signal temperatures as shown in Table 12.

The Compound 1 mono-tosylate crystalline forms of the present disclosure were prepared as described above, and subjected to melting point range analysis as discussed above. The melting point ranges are set forth in Table 13.

TABLE 13
Melting Point Ranges For Compound 1 Mono-
Tosylate Crystalline Forms 1-2 and 4-6.

| Compound 1 mono-tosylate | |
|---|---|
| crystalline form | Melting point Range (° C.) |
| Amorphous | 184-202 |
| Form 1 | 114-131 |
| Form 2 | 123-131 |
| Form 4 | 182-200 |
| Form 5 | 183-199 |
| Form 6 | 196-202 |

In some embodiments, the Compound 1 mono-tosylate crystalline form comprises a melting point range as shown in Table 13.

The Compound 1 mono-tosylate crystalline forms of the present disclosure were prepared as described above, and subjected to FT-JR analysis as discussed above. The FT-JR signals are set forth in Table 14.

TABLE 14
FT-IR Signals For Compound 1 Mono-Tosylate
Crystalline Forms 1-2 and 4-6.

| | | | |
|---|---|---|---|
| Wavenumber | % | Wavenumber | % |
| (cm⁻¹) | Transmittance | (cm⁻¹) | Transmittance |

Form 1

| | | | |
|---|---|---|---|
| 3393.7 | 13.9 | 1120.9 | 28.5 |
| 3257.4 | 14.5 | 1052.1 | 16.6 |
| 3027.8 | 15.9 | 1032.6 | 28.4 |
| 2917.6 | 16.1 | 1008.6 | 30.8 |
| 2876.3 | 15.9 | 972.1 | 18.2 |
| 2297.8 | 12.8 | 958.3 | 17.9 |
| 1920.4 | 12.3 | 930.4 | 15.7 |
| 1682.8 | 16.1 | 885.2 | 18.7 |
| 1627.8 | 17.3 | 810.2 | 23.9 |
| 1603.3 | 16.9 | 772.4 | 16.7 |
| 1570.3 | 17.3 | 734.7 | 18.7 |
| 1538.7 | 21.8 | 708.1 | 17.4 |
| 1488.2 | 19.9 | 677.6 | 33.3 |
| 1473.6 | 19.1 | 639.6 | 20.7 |
| 1401.8 | 18.9 | 597.9 | 18.2 |
| 1365.8 | 18.4 | 563.6 | 37.5 |
| 1281.7 | 14.8 | 510.7 | 23.3 |
| 1169.5 | 29.6 | 468.1 | 25.8 |

Form 2

| | | | |
|---|---|---|---|
| 3421.7 | 17.7 | 1332.6 | 15.3 |
| 3272.0 | 18.0 | 1284.7 | 18.3 |
| 3060.2 | 19.4 | 1167.6 | 44.6 |
| 3028.7 | 19.5 | 1118.2 | 48.4 |
| 2960.1 | 19.2 | 1032.5 | 42.8 |
| 2917.3 | 19.3 | 1008.2 | 46.7 |
| 2856.5 | 19.3 | 887.1 | 26.1 |
| 1920.3 | 11.7 | 870.2 | 32.8 |
| 1688.6 | 19.7 | 815.1 | 34.3 |
| 1627.5 | 23.6 | 770.9 | 22.2 |
| 1605.4 | 23.9 | 709.3 | 23.9 |
| 1571.0 | 24.6 | 681.3 | 49.4 |
| 1547.6 | 30.9 | 657.9 | 32.8 |
| 1489.9 | 27.7 | 612.9 | 30.1 |
| 1473.7 | 25.4 | 564.9 | 48.4 |
| 1453.6 | 20.0 | 511.2 | 37.2 |
| 1400.9 | 24.7 | 467.1 | 40.1 |
| 1365.9 | 23.9 | | |

Form 4

| | | | |
|---|---|---|---|
| 3460.2 | 11.9 | 1172.3 | 19.9 |
| 3249.2 | 12.1 | 1120.9 | 18.8 |
| 3021.9 | 12.9 | 1033.1 | 19.7 |
| 2917.2 | 13.2 | 1009.9 | 19.9 |
| 1684.7 | 13.2 | 973.9 | 14.0 |
| 1628.9 | 13.2 | 928.4 | 12.9 |
| 1605.2 | 13.4 | 889.4 | 15.2 |
| 1570.9 | 14.2 | 808.2 | 18.3 |
| 1540.5 | 16.7 | 706.4 | 13.8 |
| 1474.4 | 14.5 | 675.5 | 22.5 |
| 1423.3 | 12.3 | 640.2 | 14.9 |
| 1402.5 | 15.3 | 565.4 | 26.1 |
| 1366.2 | 14.6 | 527.8 | 16.4 |
| 1279.9 | 12.2 | 500.9 | 16.3 |
| 1259.6 | 12.5 | 466.7 | 18.7 |
| 1193.6 | 20.1 | | |

Amorphous

| | | | |
|---|---|---|---|
| 1686.5 | 10.4 | 744.2 | 10.8 |
| 1627.4 | 10.4 | 720.2 | 10.8 |
| 1601.5 | 10.4 | 708.7 | 10.8 |
| 1542.6 | 10.6 | 680.1 | 11.9 |
| 1487.5 | 10.6 | 656.7 | 11.2 |
| 1473.4 | 10.6 | 639.5 | 11.2 |
| 1400.1 | 10.5 | 611.9 | 10.9 |
| 1362.5 | 10.6 | 599.5 | 10.9 |
| 1281.8 | 10.4 | 562.8 | 12.1 |
| 1161.9 | 11.1 | 514.9 | 11.5 |
| 1119.2 | 11.1 | 500.6 | 11.6 |
| 1030.4 | 11.1 | 467.4 | 11.7 |
| 1006.2 | 11.3 | 452.7 | 11.5 |
| 922.3 | 10.7 | 437.5 | 11.5 |
| 880.6 | 10.8 | 426.5 | 11.4 |
| 814.6 | 11.2 | 406.5 | 11.5 |

In some embodiments, the Compound 1 mono-tosylate crystalline forms described herein comprise corresponding FT-JR signals, ±0.2 wavenumber, as shown in Table 14. In some embodiments, the Compound 1 mono-tosylate crystalline form comprises FT-JR signals, ±0.2 wavenumber, having a % transmittance of at least 15%, as shown in Table 14. In some embodiments, the Compound 1 mono-tosylate crystalline form comprises FT-JR signals, ±0.2 wavenumber, having a % transmittance of at least 20%, as shown in Table 14.

### Proton NMR

As explained above, all proton NMR experiments performed and reported herein were performed on a Bruker AVIIIHD spectrometer equipped with a DCH cryoprobe operating at 500.12 MHz for protons in deuterated DMSO. Each sample analyzed was prepared to ca. 10 mM concentration.

The ¹H NMR spectrum for crystalline form 1 of the present disclosure was prepared. All protons of Compound 1 mono-tosylate were accounted for, and the presence of the mono-tosylate moiety was confirmed. Dichloromethane solvent (0.3 equivalents) as well as some residual ethyl acetate and hexane appeared to be present. The ¹H NMR pattern indicates that Compound 1 mono-tosylate crystalline form 1 of the disclosure is non-solvated, and does not rule out the possibility of being a hydrate.

The ¹H NMR spectrum for crystalline form 2 of the present disclosure was prepared. All protons of Compound 1 mono-tosylate were accounted for, and the presence of the mono-tosylate moiety was confirmed. 1.3 equivalents of 1,4-dioxane was present. This spectrum overlaps with a signal of amorphous Compound 1 mono-tosylate (attributed to 1 proton). Some residual hexane also appeared to be present. This NMR data and the XRPD of FIG. 4 indicate that Pattern 2 is likely a mono-dioxane solvate.

As explained above, Compound 1 mono-tosylate crystalline form 3 disclosed herein converts to Compound 1 mono-tosylate crystalline form 4 on drying. The ¹H NMR spectrum for crystalline form 4 was prepared. All protons of Compound 1 mono-tosylate were accounted for, and the presence of the mono-tosylate moiety was confirmed. THF (0.12 equivalents) as well as some residual hexane appeared to be present. A small, non-stoichiometric amount of THF remained in crystalline form 4, which suggests crystalline form 4 is likely anhydrous with residual THF present.

The 1H NMR spectrum for crystalline form 5 was prepared. All protons of Compound 1 mono-tosylate were accounted for, and the presence of the mono-tosylate moiety was confirmed.

The ¹H NMR spectrum for crystalline form 6 was prepared. The Compound 1 mono-tosylate was confirmed. Trace amounts of ethanol were present in the crystal, suggesting that Pattern 6 is non-solvated.

### Polarized Light Microscopy (PLM)

Compound 1 mono-tosylate crystalline forms of the present disclosure underwent PLM. The presence of crystallinity (birefringence) was determined using an Olympus BX53 microscope, equipped with cross-polarizing lenses and a Motic camera. Images were captured using Motic Images Plux 3.0 All images were recorded using the 20× objective, unless otherwise stated.

Light micrographs for Compound 1 mono-tosylate crystalline form 1 recovered from dichloromethane (DCM) were taken. The particles appeared to consist of agglomerates with no distinct morphology. Under polarized light, the particles appeared to be birefringent, indicative of a crystalline material. The particles appeared to consist of rod-like particles, and agglomerates were also present. These particles also appeared birefringent under polarized light, indicative of a crystalline material.

Light micrographs of Compound 1 mono-tosylate crystalline form 2 recovered from methylethylketone were taken. The particles did not appear to consist of a distinct morphology, but appeared birefringent under polarized light.

Light micrographs of Compound 1 mono-tosylate crystalline form 4 were taken. The particles of appeared to consist of rod-like particles. They were similar but smaller than the rod-like particles of Compound 1 mono-tosylate crystalline form 1. Agglomerates were also present. The particles appeared birefringent under polarized light.

Light micrographs of Compound 1 mono-tosylate crystalline form 5 were taken. The particles appeared to consist of small rod-like particles, irregular particles, and agglomerates, similar to what was observed for Compound 1 mono-tosylate crystalline form 4 (from which crystalline form 5 converted). The particles appeared to be birefringent under polarized light.

Light micrographs of Compound 1 mono-tosylate crystalline form 6 were taken. The particles appeared to consist of some plate-like particles, irregular particles and agglomerates. The particles appeared birefringent under polarized light, indicative of a crystalline material.

### FT-IR of Compound 1 Mono-Tosylate Crystalline Forms

As explained above, all FT-IR experiments disclosed herein were carried out on a Bruker ALPHA P spectrometer. Sufficient material was placed onto the center of the plate of the spectrometer and the spectra were obtained using the following parameters:
Resolution: 4 cm⁻¹
Background Scan Time: 16 scans Sample Scan Time: 16 scans
Data Collection: 4000 to 400 cm⁻¹
Result Spectrum: Transmittance
Software: OPUS version 6

The FT-IR spectrum for Compound 1 mono-tosylate crystalline form 1 is set forth in FIG. 2. This spectrum is consistent with the spectrum produced for amorphous Compound 1. The signal at about 1685 cm⁻¹ is due to the amide group of the compound. Signals around 1630 cm⁻¹ to around 1560 cm⁻¹ are consistent with the cyclic C═N group of the isoquinoline moiety.

The FT-IR spectrum for Compound 1 mono-tosylate crystalline form 2 is set forth in FIG. 5. As with crystalline form 1, the spectrum obtained for crystalline form 2 is consistent with the spectrum produced for amorphous Compound 1. The signal at about 1688 cm⁻¹ is due to the amide group of the compound. Signals around 1630 cm⁻¹ to around 1560 cm⁻¹ are consistent with the cyclic C═N group of the isoquinoline. Signals between about 2960 cm⁻¹ and about 2850 cm⁻¹ are likely due to the presence of 1,4-dioxane solvent.

The FT-IR spectrum for Compound 1 mono-tosylate crystalline form 4 is set forth in FIG. 8. This spectrum is consistent with that collected for amorphous Compound 1. The signal at about 1685 cm⁻¹ is due to the amide group of the compound. Signals around 1630 cm⁻¹ to around 1560 cm⁻¹ are consistent with the cyclic C═N group of the isoquinoline moiety. Signals in the fingerprint region were consistent with toluenesulfonic acid.

### Discussion

Amorphous Compound 1 mono-tosylate salt was prepared pursuant to disclosure of U.S. Pat. No. 10,316,029, and was used to perform a polymorphism study of the starting material. The study investigated 24 solvent systems (including binary mixtures) and 4 relevant crystallization conditions: thermal cycling, cooling and anti-solvent addition. During the course of the study, amorphous Compound 1 mono-tosylate salt was predominantly recovered, suggesting that it does not readily crystallize. A more distinct set of conditions was needed to crystallize tis material. No crystalline material was recovered from evaporation, cooling, or anti-solvent addition experiments. Despite this, four crystalline forms were identified during primary screening. The diffraction patterns of these potential forms did show similarities in their dominant signals and general signal positions, but also had their own distinctions. Characterization of these potential forms help indicated whether they were likely solvated, hydrate or anhydrous.

Unlike the other 3 potential crystalline forms recovered during primary screening, crystalline form 1 was observed from multiple solvent systems. Crystalline form 1 was recovered from 2-MeTHF, DCM, both post-thermal cycling and was also produced when amorphous solid recovered from MEK (post-thermal cycling) was dried at 40° C. Thermal (TG/DSC data suggested that crystalline form 1 was initially thought to be a monohydrate, based on the distinct loss of approximately 1 equivalent of water was observed during heating and a related endothermic was noted with an onset of 113° C. The onset of thermal decomposition was evident at 189° C. There was no clear presence of water observed by FT-IR, however. Furthermore, correlation of the distinct mass lost in the thermogram with the organic solvent content detected by ¹H NMR indicated that the loss on heating was likely due to entrapped organic solvent rather than water from a hydrate. As such, crystalline form 1 was likely an anhydrous form.

Crystalline form 2 was produced from 1,4-dioxane only (post thermal cycling), which indicated that it was likely a solvate from. Thermal (TG/DSC) and ¹H NMR analysis suggested that crystalline form 2 was a mono-dioxane solvate.

Crystalline form 3 was produced from THF only (post thermal cycling) and was found to convert to crystalline form 4 on drying at 40° C. for approximately 24 hours. Crystalline form 3 was thus considered to be a weak THF solvate. Crystalline form 4 appeared to contain a non-stoichiometric amount of entrapped THF (by TG/DSC and ¹H NMR) from the likely desolvation of crystalline form 3. Thus, crystalline form 4 appeared to be a non-hydrous form of Compound 1 mono-tosylate that was accessed only via desolvation of crystalline form 3. Crystalline forms 1, 2, and 4 appeared to decompose at a similar temperature (about 182° C. to about 189° C.). Although not obligated to explain how any crystalline form of Compound 1 mono-tosylate of the present disclosure is formed, and indeed under no obligation to do so, it is possible that solvated/hydrated forms of Compound 1 mono-tosylate convert to the same anhydrous form prior to melting/decomposing or they are all rendered amorphous on heating as the received amorphous material all appeared to decompose at a similar temperature (about 184° C. by TG/DSC).

While performing competitive slurry experiments on crystalline forms 1, 4 and 5 in ethanol (process solvent) at two temperatures (ambient and 60° C.), an additional crystalline form 6 was uncovered. Thermal and NMR analyses of crystalline form 6 showed little to no EtOH present, which demonstrated crystalline form 6 is not an ethanol solvate. Moreover, no loss in mass on heating crystalline form 6 was observed (prior to melting/decomposition). Thus, crystalline form 6 is a stable anhydrous form. Crystalline form 6 can be re-prepared with seeding a saturated solution of EtOH. The melting point of crystalline form 6 is about 196° C., which is higher than the melting points of the other crystalline forms of Compound 1 mono-tosylate salt of the present disclosure. A flow diagram of the formation of crystalline forms 1-6 is set forth in FIG. 14.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

What is claimed is:

1. A solid form of Compound 1 mono-tosylate, selected from form 1 of Compound 1 mono-tosylate, form 2 of Compound 1 mono-tosylate, form 3 of Compound 1 mono-tosylate, form 4 of Compound 1 mono-tosylate, form 5 of Compound 1 mono-tosylate, or form 6 of Compound 1 mono-tosylate.

2. The solid form of claim 1, wherein the solid form is a crystalline form 1 of Compound 1 mono-tosylate characterized by data selected from one or more of the following:
(i) an x-ray powder diffraction (XRPD) pattern having two or more signals selected, independently, from 6.1, 15.8, 18.8, or 23.9 degrees 2θ±0.2 degrees 2θ;
(ii) an XRPD pattern substantially as depicted in FIG. 1;
(iii) an XRPD pattern having two or more signals selected, independently, from 6.1, 15.8, 18.8, or 23.9 degrees 2θ±0.2 degrees 2θ, and having one, two, three, four, five, six, or seven additional signals selected from 5.2, 16.5, 17.8, 19.5, 20.7, 22.8, or 23.1 degrees 2θ±0.2 degrees 2θ; and
(iv) combinations of any of (i)-(iii).

3. The solid form of claim 1, wherein the solid form is a crystalline form 2 of Compound 1 mono-tosylate characterized by data selected from one or more of the following:
(i) an x-ray powder diffraction (XRPD) pattern having two or more signals selected, independently, from 5.9, 15.5, or 16.4 degrees 2θ±0.2 degrees 2θ;
(ii) an XRPD pattern substantially as depicted in FIG. 4;
(iii) an XRPD pattern having two or more signals selected, independently, from 5.9, 15.5, or 16.4 degrees 2θ±0.2 degrees 2θ, and having one or two additional signals selected from 5.4 or 21.6 degrees 2θ±0.2 degrees 2θ; and
(iv) combinations of any of (i)-(iii).

4. The solid form of claim 1, wherein the solid form is a crystalline form 3 of Compound 1 mono-tosylate characterized by data selected from one or more of the following:
(i) an x-ray powder diffraction (XRPD) pattern having two or more signals selected, independently, from 6.9, 18.1, or 21.8, degrees 2θ±0.2 degrees 2θ;
(ii) an XRPD pattern substantially as depicted in FIG. 18;
(iii) an XRPD pattern having two or more signals selected, independently, from 6.9, 18.1, or 21.8, degrees 2θ±0.2 degrees 2θ, and having one or two additional signals selected from 19.2 or 25.3 degrees 2θ±0.2 degrees 2θ; and
(iv) combinations of any of (i)-(iii).

5. The solid form of claim 1, wherein the solid form is a crystalline form 4 of Compound 1 mono-tosylate characterized by data selected from one or more of the following:
(i) an x-ray powder diffraction (XRPD) pattern having two or more signals selected, independently, from 6.0, 17.7, 18.0, or 19.4, degrees 2θ±0.2 degrees 2θ;
(ii) an XRPD pattern substantially as depicted in FIG. 7;
(iii) an XRPD pattern having two or more signals selected, independently, from 6.0, 17.7, 18.0, or 19.4, degrees 2θ±0.2 degrees 2θ, and having one, two, or three additional signals selected from 16.0, 19.7, or 25.2 degrees 2θ±0.2 degrees 2θ; and
(iv) combinations of any of (i)-(iii).

6. The solid form of claim 1, wherein the solid form is a crystalline form 5 of Compound 1 mono-tosylate characterized by data selected from one or more of the following:
(i) an x-ray powder diffraction (XRPD) pattern having two or more signals selected, independently, from 6.2, 7.3, 19.8, or 23.4 degrees 2θ±0.2 degrees 2θ;
(ii) an XRPD pattern substantially as depicted in FIG. 10;
(iii) an XRPD pattern having two or more signals selected, independently, from 6.2, 7.3, 19.8, or 23.4 degrees 2θ±0.2 degrees 2θ degrees, and having one, two, three, four, five or six additional signals at 6.6, 10.6, 14.6, 15.9, 21.2, and 27.9 degrees 2θ±0.2 degrees 2θ; and
(iv) combinations of any of (i)-(iii).

7. The solid form of claim 1, wherein the solid form is a crystalline form 6 of Compound 1 mono-tosylate characterized by data selected from one or more of the following:
(i) an x-ray powder diffraction (XRPD) pattern having two or more signals selected, independently, from 9.7, 11.2, 12.8, 14.5, or 23.0 degrees 2θ±0.2 degrees 2θ;
(ii) an XRPD pattern substantially as depicted in FIG. 12;
(iii) an XRPD pattern having two or more signals selected, independently, from 9.7, 11.2, 12.8, 14.5, or 23.0 degrees 2θ±0.2 degrees 2θ, and having one, two, three, or four additional signals selected from 10.9, 20.0, 22.2, or 25.6 degrees 2θ±0.2 degrees 2θ; and
(iv) combinations of any of (i)-(iii).

8. The solid form of claim 1, having an XRPD pattern substantially as shown in FIG. 1, FIG. 4, FIG. 18, FIG. 7, FIG. 10, or FIG. 12.

9. The solid form of claim 1, having a differential scanning calorimetry (DSC) thermogram substantially as shown in FIG. 3, FIG. 6, FIG. 9, FIG. 11, or FIG. 13.

10. The solid form of claim 1, having a thermogravimetric analysis (TGA) substantially as shown in FIG. 3, FIG. 6, FIG. 9, FIG. 11, or FIG. 13.

11. The solid form of claim 1, having a melting point range of about 114-131, 123-131, 182-200, 183-199, or 196-202±about 3° C.

12. The solid form of claim 1, having a DSC signal of about 52.9, 128.9, 201.3, 131.0, 199.7, 115.3, 199.9, 186.8, 198.7, 223.8, or 201.5±about 3° C.

13. The solid form of claim 1, prepared by a process comprising crystallization from a solvent.

14. A pharmaceutical composition for treating an ocular disease or disorder in a subject, comprising the solid form of any of claims 1-13 or any combination thereof, and at least one pharmaceutically acceptable excipient.

15. The pharmaceutical composition of claim 14, wherein the pharmaceutical composition is formulated for intravitreal administration to an eye of the subject.

16. The pharmaceutical composition of claim 14 or 15, wherein the at least one pharmaceutically acceptable excipient comprises a biodegradable polymer matrix.

17. The pharmaceutical composition of claim 16, wherein the biodegradable polymer matrix comprises a mixture of a first polymer and a second polymer, wherein:
(a) the first polymer is a biodegradable polyesteramide polymer; and
(b) the second polymer is at least one biodegradable poly(D,L-lactide) polymer, at least one biodegradable poly (D,L-lactide-co-glycolide) polymer, or any combination of at least one biodegradable poly(D,L-lactide) polymer and at least one biodegradable poly (D,L-lactide-co-glycolide) polymer thereof.

18. The pharmaceutical composition of claim 17, wherein the biodegradable polymer matrix is a mechanical blend of the first polymer and the second polymer.

19. The pharmaceutical composition of any of claims 16-18, wherein the pharmaceutical composition comprises at least about 50 weight % of the biodegradable polymer matrix.

20. The pharmaceutical composition of any of claims 18-19, wherein the at least one (D,L-lactide) polymer is an acid end-capped biodegradable poly(D,L-lactide) homopolymer, or an ester end-capped poly(D,L-lactide) homopolymer, or any combination thereof.

21. The pharmaceutical composition of any of claims 18-20, wherein the at least one poly(D,L-lactide-co-glycolide) polymer is an ester end-capped biodegradable poly(D,L-lactide-co-glycolide) copolymer, or an acid-capped biodegradable poly(D,L-lactide-co-glycolide) copolymer, or any combination thereof.

22. The pharmaceutical composition of any of claims 18-21, wherein the biodegradable polyesteramide polymer is a homopolymer that comprises structure (I): 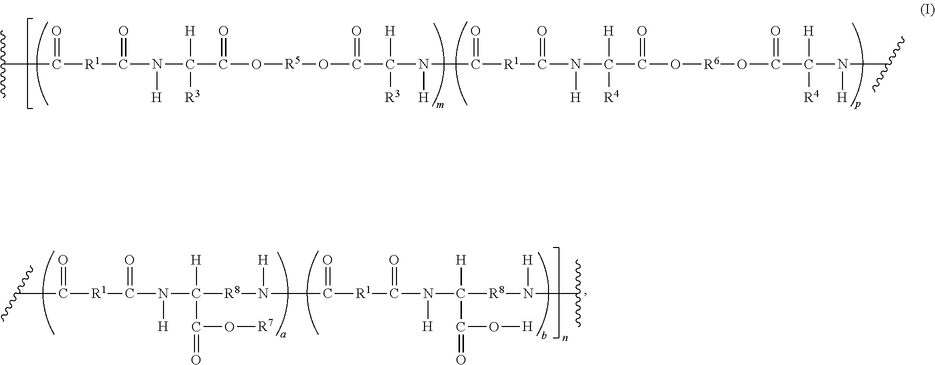
or a salt thereof,
wherein
m+p varies from 0.9-0.1 and a+b varies from 0.1 to 0.9;
m+p+a+b=1, wherein one of m or p could be 0;
n varies from 5 to 300 and wherein a is at least 0.01, b is at least 0.015 and the ratio of a to b (a:b) is from 0.1:9 to 0.85:0.15, wherein the m unit and/or p unit, and the a and b units, are randomly distributed;
R¹ is independently selected from (C₂-C₂₀)alkyl;
R³ and R⁴ in a single backbone unit m or p, respectively, are independently selected from hydrogen, (C₁-C₆)alkyl, (C₂-C₆)alkenyl, (C₂-C₆)alkynyl, (C₆-C₁₀)aryl, (C₁-C₆ alkyl, —(CH₂)SH, —(CH₂)₂S(CH)₃, (CH₃)₂—CH—CH₂—, —CH(CH₃)₂, —CH(CH₃)—CH₂—CH₃, —CH₂—C₆H₅, —(CH₂)₄—NH₂, and mixtures thereof;
R⁵ is independently selected from (C₂-C₂₀)alkyl, (C₂-C₂₀)alkenylene;
R⁶ is selected from bicyclic-fragments of 1,4:3,6-dianhydrohexitols of structural formula (II): 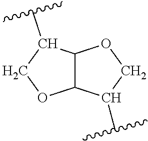 [SMILES: *C1COC2C(*)COC12]
R⁷ is independently selected from the group consisting of (C₆-C₁₀) aryl, (C₁-C₆)alkyl or a protecting group; and
R⁸ is —(CH₂)₄—.

23. The pharmaceutical composition of any of claims 18-22, wherein the biodegradable polyesteramide polymer is a homopolymer that comprises structure (II): 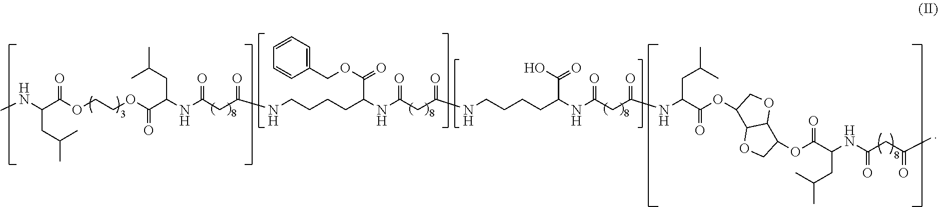
or a salt thereof.

24. The pharmaceutical composition of any of claims 14-23, wherein the pharmaceutical composition is in the form of an ocular implant.

25. The pharmaceutical composition of any of claims 14-24, wherein the pharmaceutical composition is formulated to release at least one crystalline form of Compound 1 mono-tosylate in a substantially linear manner for at least about 1 month to at least about 6 or 12 months.

26. The pharmaceutical composition of any of claims 14-25, wherein the ocular disease or disorder comprises glaucoma, a neurodegenerative disease or disorder, ocular hypertension, an inflammatory disease or disorder, or any combination thereof.

27. The pharmaceutical composition of claim 26, wherein the neurodegenerative disease or disorder comprises diabetic eye disease, wet age-related macular degeneration, dry aged-related macular degeneration, inflammation, dry eye, or any combination thereof.

28. The pharmaceutical composition of claim 26, wherein the inflammatory disease or disorder comprises uveitis, a corneal ulcer, endophthalmitis, an autoimmune disease of the cornea or ocular surface, an ophthalmic manifestation of HIV disease, or ocular herpes

29. A method of treating an ocular disease or disorder in a subject in need thereof, comprising administering to the subject a therapeutically effective amount of the solid form of any of claims 1-13, or any combination thereof.

30. A method of treating an ocular disease or disorder in a subject in need thereof, comprising administering to the subject a therapeutically effective amount of the pharmaceutical composition of any of claims 14-28.

31. The method of claim 29 or 30, wherein administering to the subject comprises administering to the vitreous humor of an eye of the subject.

32. The method of any of claims 29-31, wherein the ocular disease or disorder comprises glaucoma, a neurodegenerative disease or disorder, ocular hypertension, an inflammatory disease or disorder, or any combination thereof.

33. The method of claim 32, wherein the neurodegenerative disease or disorder comprises diabetic eye disease, wet age-related macular degeneration, dry aged-related macular degeneration, inflammation, dry eye, or any combination thereof.

34. The method of claim 32, wherein the inflammatory disease or disorder comprises uveitis, a corneal ulcer, endophthalmitis, an autoimmune disease of the cornea or ocular surface, an ophthalmic manifestation of HIV disease, ocular herpes.
